(19) United States
(12) Patent Application Publication
(10) Pub. No.: US 2025/0084473 A1
(43) Pub. Date: Mar. 13, 2025

(54) METHODS FOR SAMPLE PREPARATION

(71) Applicant: Autonomous Medical Devices Incorporated, Santa Ana, CA (US)

(72) Inventor: Regis PEYTAVI, Laguna Niguel, CA (US)

(21) Appl. No.: 18/961,229

(22) Filed: Nov. 26, 2024

Related U.S. Application Data
(63) Continuation of application No. PCT/US2023/067879, filed on Jun. 2, 2023.
(60) Provisional application No. 63/348,750, filed on Jun. 3, 2022.

(51) Int. Cl. C12Q 1/6869; C12Q 1/6806; C12Q 1/686

(52) U.S. Cl. CPC C12Q 1/6869; C12Q 1/6806; C12Q 1/686

(57) ABSTRACT

Provided herein are methods for preparing a sample comprising a nucleic acid. In some embodiments, the method comprises heating the sample above its boiling point to rapidly extract nucleic acid from biological entities present in the sample.

### CROSS-REFERENCE TO RELATED APPLICATIONS

This application is a continuation of International Application No. PCT/US2023/067879, filed Jun. 2, 2023, which claims the benefit of U.S. Provisional Application No. 63/348,750, filed Jun. 3, 2022, which is herein incorporated by reference in its entirety for all purposes.

### BACKGROUND

Two methods for extraction and purification of nucleic acids are primarily used during sample preparation for nucleic acid analysis. The first method comprises the purification of the nucleic acid from other molecular species present in a sample via extraction. Such methods often comprise phenol/chloroform extraction followed by ethanol precipitation, use of chaotropic lysis agent followed by ion exchange resin, or use of a silica resin. These methods provide a high-quality nucleic acid (e.g., highly pure nucleic acid), but are time consuming and involved numerous complicated steps. The second method is often referred to as extraction-free, and this method uses physical, enzymatic, or chemical means to lyse the cell or microorganisms and amplify the nucleic acid without undergoing further purification. The second method is simpler and usually faster than the first method, but provides nucleic acid preparation of lesser quality than the first method.

To effectively amplify the nucleic acid in the second method, amplification inhibitors (e.g., PCR inhibitors) present in the sample must be neutralized so that molecular amplification of the nucleic acid can occur prior to detection. Further, the nucleic acid must not be degraded, so that it can be amplified and detected. For example, metal ions present in cells and/or tissues inhibit some amplification enzymes and can lead to nucleic acid degradation. In addition, natural enzymes present in biological samples can either inhibit molecular amplification (e.g., proteases) or degrade the nucleic acid (e.g., nucleases). Current means for inactivating these amplification inhibitors, e.g., PCR inhibitors, rely on dilution of at least 10 times of the sample, and this reduces the sensitivity of the overall assay. Further, storage of diluent in a fluidic cartridge creates complexity of the overall manufacturing and reduces the shelf life of a point of care device. Thus, a simpler, faster method for effective sample processing prior to nucleic acid amplification and detection is highly desirable.

### SUMMARY

In one aspect, the present disclosure provides a method of analyzing a nucleic acid, the method comprising: (a) heating a treatment sample comprising a nucleic acid in a closed heating chamber in a hyperbaric condition from a first temperature to a second temperature above 100 degrees Celsius over a ramp time, thereby producing a heat-treated sample; and (b) analyzing the nucleic acid from the heat-treated sample.

In some embodiments, the treatment sample comprises one or more reagents selected from the group consisting of: a chelating agent, a single stranded nucleic acid binding protein, a reducing agent, and a stabilizer.

In some embodiments, the treatment sample comprises a chelating agent, a single stranded nucleic acid binding protein, and a reducing agent.

In some embodiments, the treatment sample comprises a chelating agent, a single stranded nucleic acid binding protein, a reducing agent, and a stabilizer.

In some embodiments, the first temperature is from 4 degrees Celsius to 40 degrees Celsius and the second temperature is from 101 degrees Celsius to 160 degrees Celsius.

In some embodiments, the ramp time is from 3 to 50 seconds.

In some embodiments, heating the treatment sample occurs at a temperature ramp rate from 5 degrees Celsius per second to 50 degrees Celsius per second.

In some embodiments, the method further comprises, after step (a) and prior to step (b), maintaining the treatment sample at the second temperature for a maintenance time from 0 seconds to 120 seconds prior to a cooling time.

In some embodiments, the treatment sample comprises a bodily sample comprising the nucleic acid, wherein: i) the bodily sample is selected from the group consisting of a blood sample, a lacrimal fluid sample, a saliva sample, a mucus sample, a sputum sample, a feces sample, a cerebrospinal fluid sample, and a urine sample; and ii) the nucleic acid is not extracted, isolated, or otherwise purified from the bodily sample.

In another aspect, the present disclosure provides a method of analyzing a nucleic acid, the method comprising: (a) providing a bodily sample selected from the group consisting of a blood sample, a lacrimal fluid sample, a saliva sample, a mucus sample, a sputum sample, a feces sample, a cerebrospinal fluid sample, and a urine sample; wherein the bodily sample comprises a nucleic acid; (b) heating a treatment sample comprising the bodily sample in a closed heating chamber in a hyperbaric condition from a first temperature to a second temperature above 100 degrees Celsius over a ramp time, thereby producing a heat-treated sample; and (c) analyzing the nucleic acid from the heat-treated sample; wherein the nucleic acid is not extracted, isolated, or otherwise purified from the bodily sample.

In some embodiments, the bodily sample is a saliva sample or a mucus sample.

In some embodiments, the bodily sample is collected via a nasopharyngeal swab, a cervical swab, or a nasal swab from a human subject.

In some embodiments, the bodily sample comprises a pathogen or a portion thereof.

In some embodiments, the pathogen or portion thereof is selected from the group consisting of a virus or a portion thereof, a bacterium or a portion thereof, a protozoon or a portion thereof, a yeast or a portion thereof, and a fungus or a portion thereof.

In some embodiments, the pathogen is a virus or a portion thereof.

In some embodiments, the virus is SARS-CoV2.

In some embodiments, the treatment sample comprises an unlysed cell comprising the nucleic acid, and wherein the nucleic acid is not extracted, isolated, or otherwise purified from the heat-treated sample prior to analyzing the nucleic acid from the heat-treated sample.

In another aspect, the present disclosure provides a method of analyzing a nucleic acid, the method comprising: (a) heating a treatment sample comprising an unlysed cell comprising a nucleic acid in a closed heating chamber in a hyperbaric condition from a first temperature to a second temperature above 100 degrees Celsius, thereby producing a heat-treated sample; and (b) analyzing the nucleic acid from the heat-treated sample; wherein the nucleic acid is not extracted, isolated, or otherwise purified from the heat-treated sample prior to step (b).

In some embodiments, the unlysed cell is selected from the group consisting of a bacterial cell, a fungus cell, and a mammalian cell.

In some embodiments, the treatment sample comprises one or more reagents selected from the group consisting of: a chelating agent, a single stranded nucleic acid binding protein, a reducing agent, and a stabilizer.

In another aspect, the present disclosure provides a method of analyzing a nucleic acid, the method comprising: (a) heating a treatment sample comprising a nucleic acid in a closed heating chamber in a hyperbaric condition from a first temperature to a second temperature above 100 degrees Celsius, thereby producing a heat-treated sample; and (b) analyzing the nucleic acid from the heat-treated sample; wherein the treatment sample comprises one or more reagents selected from the group consisting of a chelating agent, a single stranded nucleic acid binding protein, a reducing agent, and a stabilizer.

In some embodiments, the treatment sample comprises at least two reagents selected from the group consisting of: a chelating agent, a single stranded nucleic acid binding protein, a reducing agent, and a stabilizer.

In some embodiments, the treatment sample comprises at least three reagents selected from the group consisting of: a chelating agent, a single stranded nucleic acid binding protein, a reducing agent, and a stabilizer.

In some embodiments, the treatment sample comprises a chelating agent, a single stranded nucleic acid binding protein, and a reducing agent.

In some embodiments, the treatment sample comprises a chelating agent, a single stranded nucleic acid binding protein, a reducing agent, and a stabilizer.

In some embodiments, the chelating agent has a concentration of from 5% to 25% weight by volume of the treatment sample, the single-stranded nucleic acid binding protein has a concentration from 0.1 μM to 2 μM in the treatment sample, the reducing agent has a concentration from 0.1 mM to 2 mM in the treatment sample, or the stabilizer has a concentration from 500 ng/mL to 10 mg/mL in the treatment sample.

In some embodiments, the chelating agent is an insoluble chelating agent.

In some embodiments, the insoluble chelating agent comprises a styrene divinylbenzene co-polymer.

In some embodiments, the chelating agent is a soluble chelating agent.

In some embodiments, the soluble chelating agent comprises EDTA.

In some embodiments, the stabilizer is bovine serum albumin or gelatin.

In some embodiments, the nucleic acid is DNA.

In some embodiments, the nucleic acid is RNA.

In some embodiments, the nucleic acid is selected from the group consisting of a viral nucleic acid, a bacterial nucleic acid, a protozoan nucleic acid, a eukaryotic nucleic acid, and a fungal nucleic acid.

In some embodiments, the nucleic acid is a viral nucleic acid.

In some embodiments, the treatment sample comprises from 50 copies/mL to 10⁹ copies/mL of the nucleic acid.

In some embodiments, the treatment sample has a volume from 100 μL to 5 mL.

In some embodiments, the treatment sample has a pH from about 8.0 to about 12.0.

In some embodiments, the treatment sample further comprises a protease.

In some embodiments, the treatment sample further comprises a nuclease inhibitor.

In some embodiments, the treatment sample is heated with a heat source.

In some embodiments, the heat source comprises an induction heater, a heating element, or a microwave.

In some embodiments, the heat source comprises an induction heater.

In some embodiments, the closed heating chamber comprises a thermally conductive material.

In some embodiments, the closed heating chamber comprises a ferromagnetic material.

In some embodiments, the nucleic acid is not substantially degraded after the heating.

In some embodiments, the method further comprises amplifying the nucleic acid by polymerase chain reaction (PCR) after the heating, thereby producing a PCR product.

In some embodiments, the method further comprises detecting the PCR product.

In some embodiments, the PCR product is detectable following a lower number of molecular amplification cycles than would be required in the absence of the heating of the treatment sample.

In some embodiments, the PCR product is detectable following a lower number of molecular amplification cycles than would be required as compared to heating the treatment sample at a temperature below 100 degrees Celsius.

In some embodiments, the PCR product is detectable after the nucleic acid is amplified using from 10 to 55 molecular amplification cycles.

In some embodiments, the PCR product is detectable following from 28 to 35 molecular amplification cycles.

In some embodiments, analyzing the nucleic acid comprises detecting the nucleic acid, sequencing the nucleic acid, or genotyping the nucleic acid.

In some embodiments, analyzing the nucleic acid comprises detecting the nucleic acid via fluorescence detection.

In some embodiments, heating the treatment sample occurs at a temperature ramp rate from 5 degrees Celsius per second to 20 degrees Celsius per second.

In another aspect, the present disclosure provides a method of inactivating a molecular amplification inhibitor in a treatment sample comprising a nucleic acid, the method comprising: heating a treatment sample comprising: i) a nucleic acid and ii) a plurality of molecular amplification inhibitors in a closed heating chamber in a hyperbaric heating condition from a first temperature to a second temperature over a ramp time above 100 degrees Celsius, thereby inactivating a molecular amplification inhibitor of the plurality of amplification inhibitors and producing a heat-treated sample, wherein the nucleic acid is not substantially degraded.

In some embodiments, the first temperature is from 4 degrees Celsius to 40 degrees Celsius and the second temperature is from 101 degrees Celsius to 160 degrees Celsius.

In some embodiments, the method further comprises maintaining the treatment sample at the second temperature for a maintenance time prior to a cooling time.

In some embodiments, the maintenance time is from 0 seconds to 120 seconds.

In some embodiments, the method inactivates at least 70% of the plurality of molecular amplification inhibitors.

In some embodiments, the method inactivates at least 90% of the plurality of molecular amplification inhibitors.

In some embodiments, the treatment sample comprises a bodily sample comprising the nucleic acid, wherein: i) the bodily sample is selected from the group consisting of a blood sample, a lacrimal fluid sample, a saliva sample, a mucus sample, a sputum sample, a feces sample, a cerebrospinal fluid sample, and a urine sample; and ii) the nucleic acid is not extracted, isolated, or otherwise purified from the bodily sample.

In some embodiments, the bodily sample is a saliva sample.

In some embodiments, the bodily sample is a mucus sample.

In some embodiments, the bodily sample is collected from an animal or human subject.

In some embodiments, the bodily sample is collected via nasopharyngeal swab, cervical swab, or nasal swab from the animal or human subject.

In some embodiments, the treatment sample comprises an unlysed cell comprising the nucleic acid.

In some embodiments, the treatment sample comprises one or more reagents selected from the group consisting of: a chelating agent, a single stranded nucleic acid binding protein, a reducing agent, and a stabilizer.

In some embodiments, the treatment sample comprises at least two reagents selected from the group consisting of: a chelating agent, a single stranded nucleic acid binding protein, a reducing agent, and a stabilizer.

In some embodiments, the treatment sample comprises at least three reagents selected from the group consisting of: a chelating agent, a single stranded nucleic acid binding protein, a reducing agent, and a stabilizer.

In some embodiments, the treatment sample comprises a chelating agent, a single stranded nucleic acid binding protein, and a reducing agent.

In some embodiments, the treatment sample comprises a chelating agent, a single stranded nucleic acid binding protein, a reducing agent, and a stabilizer.

In some embodiments, the chelating agent has a concentration from 5% to 25% weight by volume of the treatment sample, the single-stranded nucleic acid binding protein has a concentration from 0.1 μM to 2 μM in the treatment sample, the reducing agent has a concentration from 0.1 mM to 2 mM in the treatment sample, or the stabilizer has a concentration from 500 ng/mL to 10 mg/mL in the treatment sample.

In some embodiments, the treatment sample has a pH from about 8.0 to about 12.0.

In some embodiments, the treatment sample further comprises a protease.

In some embodiments, the treatment sample further comprises a nuclease inhibitor.

In some embodiments, heating the treatment sample occurs at a temperature ramp rate from 5 degrees Celsius per second to 50 degrees Celsius per second.

In another aspect, the present disclosure provides a method of analyzing a nucleic acid, the method comprising: (a) heating a treatment sample comprising a nucleic acid in a closed heating chamber in a hyperbaric condition from a first temperature to a second temperature above 100 degrees Celsius over a ramp time, thereby producing a heat-treated sample; and (b) analyzing the nucleic acid from the heat-treated sample; wherein the heating occurs at a temperature ramp rate from 5 degrees Celsius per second to 50 degrees Celsius per second.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 shows the results of the RT-PCR experiment following one method of the present disclosure (utilizing high temperature for a 40 seconds, 60 seconds, 80 seconds, or 100 seconds) and a traditional method of sample preparation (heating to 98 degrees Celsius for 300 sec). The results demonstrate that high PCR efficiency (indicated by low average cycle threshold) is achieved in much shorter time (e.g., 60 seconds) compared to the traditional method (i.e., 300 seconds).

FIG. 2 shows that treatment of a nasal swab sample comprising inactivated SARS-CoV-2 virus with a method of the present disclosure (e.g., hyperbaric heating to 130 degrees Celsius) resulted in an increase in PCR efficiency, as indicated by a lower average cycle threshold.

FIG. 3 shows that sample preparation using a method of the present disclosure provides similar PCR detection and sensitivity as compared to the sample preparation using Qiagen Viral RNA kit.

FIG. 4 shows that methods of the present disclosure can utilize a variety of additives in addition to or as an alternative to a chelating agent, including a reducing agent, a RNAse inhibitor, or a combination thereof.

FIG. 5 shows that sample preparation using a method of the present disclosure provides similar PCR detection and sensitivity for a variety of nucleic acid containing samples as compared to the sample preparation using Qiagen Viral RNA kit or Qiagen DNeasy UltraClean Microbe DNA Kit.

FIGS. 6A-6C illustrate multiple views of a hyperbaric heating vessel. FIG. 6A shows a side view, FIG. 6B shows a top view, and FIG. 6C shows a bottom view of the hyperbaric heating vessel.

FIGS. 7A-7B illustrate a hyperbaric heating vessel, wherein the sample inlet is a duckbill valve. FIG. 7A shows a side view, and FIG. 7B shows a cut view of the hyperbaric heating vessel and the duckbill valve.

FIG. 8 provides a photograph of an induction heating platform.

FIG. 9 illustrates a closed hyperbaric heating chamber comprising an outlet channel housed within a hyperbaric heating vessel.

FIG. 10 provides photographs of a bottom view of a disclosed hyperbaric heating vessel illustrating a laser valve sealing the disclosed outlet channel.

FIGS. 11A-11B illustrate the movement the sample during sample injection and sample release and zoom-in views of the laser valve. FIG. 11A illustrates the movement of the sample during sample injection and provides a zoom-in view of the outlet channel and closed laser valve. FIG. 11B illustrates the movement of the sample during sample release and provides a zoom-in view of the outlet channel and the sample movement through the open laser valve.

### DETAILED DESCRIPTION

Provided herein are methods for nucleic acid sample preparation using temperature above 100 degrees Celsius to rapidly liberate high-quality nucleic acid from microorganisms or eukaryotic cells in a form that is ready to use for downstream applications. Briefly, a sample collected from a subject (e.g., a bodily sample) is heated (e.g., hyperbaric heating) for a short period of time (e.g., less than 60 seconds) at a temperature above 100 degrees Celsius (e.g., 130 degrees Celsius) to lyse cells and/or viruses in the sample, freeing the nucleic acids (both DNA and RNA) present therein. The methods described in the present disclosure not only provide an efficient technique to free nucleic acids from other components in the biological sample (e.g., proteins or other cellular components), but these methods can also destroy amplification inhibitors, e.g., PCR inhibitors, that are present in the biological sample. Thus, the methods described in the present disclosure provide nucleic acid samples that can be used for downstream applications such as (e.g., molecular amplification, nucleic acid detection, nucleic acid sequencing, etc.) without the need of further dilution of the sample. In some cases, the methods described in the present disclosure provide nucleic acids samples that can be used for molecular amplification and/or PCR analysis without the need to boost DNA polymerase, reverse transcriptase or RNAse inhibitor concentration. In addition, the method is as efficient as more cumbersome nucleic acid extraction methods to reach a high sensitivity with PCR in less than 10 minutes thermocycling when using an ultrafast thermocycler. As such, methods of the present disclosure can save time and resources, such as the use of purification kits and expensive enzymes, in the sample preparation step.

Although some nucleic acids, such as, for example RNA, are known to be degraded at high temperature, the present disclosure provides the surprising result that nucleic acids (including both DNA and RNA) can be efficiently amplified following heating to temperatures above 100 degrees Celsius. This finding is particularly surprising with respect to RNA, which has been widely reported to be unstable at elevated temperatures. In some aspects of the present disclosure, the sample is added to a device or a vessel (e.g., a sealed vessel) that can sustain high steam pressure, and the sample is heated to temperatures above 100 degrees Celsius while inside the device or vessel (e.g., hyperbaric heating). In some embodiments, prior to the heating step, the vessel is sealed, thereby preventing steam from escaping during the hyperbaric heating step and facilitating pressure build up within the vessel. In some embodiments, the pressure inside the vessel builds up, for example, to about 15 to 40 PSI (including the atmospheric pressure). Further, the sealed vessel can prevent the sample solution (e.g., aqueous sample solution) from boiling, thereby allowing the sample solution to reach temperatures significantly above 100 degrees Celsius (e.g., 130 degrees Celsius). Prevention of the sample solution from boiling can result in cell and/or viral lysis and the neutralization of enzyme inhibitors present in the sample without damaging nucleic acids (DNA or RNA).

Further, in some aspects of the present disclosure, an additive is added to the sample (e.g., prior to heating, during heating, or after heating). For example, a chelating agent, such as, Chelex, can be added to the sample prior to hyperbaric heating. In some embodiments, the additive is a chelator, a reducing agent, a single-stranded nucleic acid binding protein, an RNAse inhibitor, or a combination thereof. In some embodiments, a chelator is not added to the sample (e.g., when a reducing agent and/or an RNAse inhibitor is added to the sample, the chelating agent can be omitted in the sample preparation). The chelating agent can be present during heating or can be used upfront the heating step and remove before heat or can be omitted all together. Even at the high temperatures used in the methods of the present disclosure, such resins sequester metal ions that would otherwise interfere with nucleic acid amplification and/or detection. In some embodiments, the pH is acidic (pH 5). In some other embodiments, the pH is close to neutral. In some embodiments, the pH of the sample is basic (e.g., a pH of 8 or higher), which, at the temperatures utilized in the present disclosure, help neutralize RNA nuclease activity. Surprisingly, even under basic conditions and high temperatures the present disclosure provides a method of sample preparation that does not result in significant degradation of the nucleic acid, even for RNA. In some embodiments, a thermostable single stranded nucleic acid binding protein (SSB) is added to the sample mixture prior to heating to further protect the nucleic acid (e.g., RNA) during the heating step, even if the pH is neutralized to a pH where nuclease enzymes are active.

RNA nuclease can sometimes survive harsh heating conditions. Thus, in some embodiments, an additive, such as a reducing agent, e.g., DTT or TCEP, either with or without a chelating agent, is added to completely neutralize RNA nuclease using a method of the present disclosure.

The present disclosure provides sample preparation methods that provide high quality of nucleic acid sample preparation, and the nucleic sample preparation from this method can readily be used for downstream application such as molecular amplification and detection. The entire process may use less time and shorter workflow compared with conventional sample processing methods, providing a more efficient method for nucleic acid sample preparation.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing genetic material. The biological entity can be a plant, animal, or microorganism, including, for example, bacteria, viruses, fungi, and protozoa. The subject can be tissues, cells, or fragments thereof, derived from a biological entity obtained in vivo or cultured in vitro. The subject can be a mammal, for example, a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the term “molecular amplification” refers to assay or method or test used to detect nucleic acids in sample. This can be used in, for example, experimental research, clinical medicine development, infectious diagnosis, gene cloning, and industrial quality control. Molecular amplification can also be used as part of a diagnostic test. The term “molecular amplification” referred herein intends to cover all methods that are designed to amplify (e.g., replicate, duplicate, etc.) nucleic acid thereby generating more copies of the nucleic acid in a sample. Molecular amplification comprises nucleic acid amplification, enzymatic amplification (e.g., PCR), isothermal amplification, and/or other alternative amplification methods that has been developed in the field.

As used herein, the terms “polymerase chain reaction” or “PCR” refers to a type of molecular or nucleic acid amplification that amplify or generate more copies of a nucleic acid template. The method of PCR can be used in conjunction with a method to detect, identify, and/or quantify the nucleic acid. The term “PCR” used herein intends to cover all different types of PCR, including, for example, sequential PCR and real-time PCR (“RT-PCR”).

As used herein, the term “biological sample” means a sample containing nucleic acids/biological agents such as clinical (e.g., cell fractions, mucus membrane, nasal swab, whole blood, plasma, serum, urine, tissue, cells, etc.), agricultural, environmental (e.g., soil, mud, minerals, water, air), food, forensic, or any other biological samples. The sample may include infectious agents, such as, for example, viral, bacterial or parasitical infectious agents. With “whole blood,” it is meant blood such as it is collected, e.g., by venous sampling, containing white and red cells, platelets, plasma, and any infectious agents that may be present. The clinical samples may be from human or animal origin. The sample analyzed can be solid or liquid in nature. It is evident when solid materials are used, these are first dissolved in a suitable solution as known in the art.

As used herein, the term “chelating agent” refers to an agent that is used to remove metal ions in solution. Chelating agents include soluble chelating agents (e.g., agents that can form a water soluble complexes with metal ions) including for example, sodium tripolyphosphate, EDTA, DTPA, NTA, citrate, and the like. Chelating agents further include insoluble chelating agents (e.g., agents that form an insoluble complex with metal ions), including, for example, sodium triphosphate, zeolite A, and the like. In some embodiments, chelating agents are used to remove metal ion impurity in the sample or inactivate and/or inhibit molecular amplification inhibitor, resulting in more efficiency in molecular amplification of the sample. Chelating agents can be used to increase quality of nucleic acid sample prior to perform molecular amplification.

As used herein, the term “reducing agent” refers to an agent that donates an electron to an electron recipient. In some embodiments, the term “reducing agent” refers to an agent that is used to reduce disulfide bound in protein. Reducing agents include, e.g., 2-Mercaptoethanol, 2-Mercaptoethylamine-HCl, TCEP, Cysteine-HCl, Dithiothreitol (DTT), TCEP-HCl, thiol-based reducing agent, Guanidine-HCl, and urea, among others. Other reducing agent that is known in the art can be used in as described in the present disclosure.

As used herein, the term “hyperbaric” or “hyperbarically” refer to the condition in which a pressure is greater or higher than normal pressure at the same altitude level.

As used herein, the term “nucleic acid” refers to DNA molecules (e.g., cDNA or genomic DNA), RNA molecules (e.g., mRNA), DNA-RNA hybrids, and analogs of DNA or RNA produced using nucleotide analogs. Nucleic acid molecules can comprise nucleotides, oligonucleotides, double-stranded DNA, single-stranded DNA, multi-stranded DNA, complementary DNA, genomic DNA, non-coding DNA, messenger RNA (mRNA), single-stranded RNA, microRNA (miRNA), and nuclear body small molecules. It may be RNA (snoRNA), ribosomal RNA (rRNA), transfer RNA (tRNA), small interfering RNA (siRNA), heteronuclear RNA (hnRNA), or small hairpin RNA (shRNA).

### Methods of Sample Preparation

The present disclosure provides methods for sample preparation for application to biological samples comprising a nucleic acid. Examples of biological samples comprising a nucleic acid may be bodily samples (e.g., saliva or mucus) or cells (e.g., a bacterial cell, a fungal cell, or a mammalian cell). The present disclosure provides methods for sample preparation of a nucleic acid in a bodily sample that do not involve extraction, isolation, or other forms of purification of the nucleic acid from the bodily sample. The present disclosure provides methods of inactivating molecular amplification inhibitors in the sample (e.g., RNAses present in saliva or mucus), thereby preparing the sample for further processing.

The present disclosure further provides methods of analyzing the nucleic acid in the biological sample comprising preparing the sample using a method of the present disclosure and subsequently analyzing the nucleic acid. The methods of sample preparation described herein may prepare the sample for molecular amplification of the nucleic acid in the sample. In some embodiments, the methods described herein may improve the efficiency of nucleic acid analysis by improving the efficiency of molecular amplification. The methods described herein may reduce the degree of nucleic acid degradation and improve the detectability of the nucleic acid or molecular amplification products thereof for analysis.

Analyzing the nucleic acid can be performed using any number of techniques known in the art (for example, sequencing the nucleic acid (e.g., sequencing by synthesis, sequencing by hybridization, nanopore sequencing, etc.), genotyping the amino acid (e.g., genotyping by hybridization, genotyping by sequencing, etc.), or detecting the nucleic acid (e.g., detection by hybridization, antibody binding, fluorescence, radioisotope detection, etc.). For example, in some embodiments, detecting the nucleic acid comprises hybridizing the nucleic acid to a fluorescently-labeled nucleic acid comprising a sequence that is complementary at least a portion of the nucleic acid using methods known in the art.

In some embodiments, analyzing the nucleic acid comprises one or more of the following: detecting the nucleic acid, sequencing the nucleic acid, and genotyping the nucleic acid. In some embodiments, analyzing the nucleic acid comprises detecting the nucleic acid. In some embodiments, analyzing the nucleic acid comprises sequencing the nucleic acid. In some embodiments, analyzing the nucleic acid comprises genotyping the nucleic acid.

In some embodiments, analyzing the nucleic acid comprises analyzing a molecular amplification product of the nucleic acid (e.g., DNA copies of RNA produced during PCR amplification of the RNA in the sample using reverse transcriptase). For example, in some embodiments, detecting the nucleic acid comprises detecting a molecular amplification product of the nucleic acid. In some embodiments, sequencing the nucleic acid comprises sequencing a molecular amplification product of the nucleic acid. In some embodiments, genotyping the nucleic acid comprises genotyping a molecular amplification product of the nucleic acid. One of ordinary skill in the art will understand that many means for analyzing the nucleic acid are known in the art, all of which are compatible with methods of the present disclosure and contemplated herein.

In some embodiments, the molecular amplification comprises enzymatic amplification. In some embodiments, the molecular amplification comprises isothermal amplification. In some embodiments, the nucleic acid amplification comprises polymerase chain reaction (“PCR”), loop mediated isothermal amplification (LAMP), nucleic acid sequence based amplification (NASBA), self-sustained sequence replication (3SR), strand displacement amplification (SDA), multiple displacement amplification (MDA), rolling cycle amplification (RCA), ligase chain reaction (LCR), helicase dependent amplification (HAD), ramification amplification method (RAM), transcription-mediated assay (TMA), Nicking enzyme amplification reaction (NEAR), Recombinase Polymerase Amplification (RPA), or whole genome amplification (WGA).

In some embodiments, the molecular amplification comprises polymerase chain reaction (“PCR”). In some embodiments, the PCR comprises reverse transcriptase-polymerase chain reaction (RT-PCR), reverse transcription-quantitative PCR (RT-qPCR), quantitative real-time PCR (qPCR), digital PCR (dPCR), digital droplet PCR (ddPCR), microfluidic PCR, multiplex PCR, variable number of tandem repeats (VNTR) PCR, asymmetric PCR, nested PCR, quantitative PCR, Hot-start PCR, touchdown PCR, assembly PCR, colony PCR, suicide PCR, co-amplification at lower denaturation temperature-PCR (COLD-PCR), rapid amplification of cDNA ends (RACE) PCR, two-tailed PCR, ligation-mediated PCR, methylation-specific PCR (MSP), InterSequence-Specific PCR (or ISSR-PCR), RNase H-dependent PCR (rhPCR), or Vectorette PCR.

In some embodiments, the nucleic acid can be analyzed after molecular amplification (e.g., PCR) of the nucleic acid. In some embodiments, the nucleic acid is analyzed as part of a downstream application. In some embodiments, the downstream application comprises molecular amplification or sequencing. In some embodiments, the downstream application comprises probe hybridization and/or detecting the probe.

In some aspects, the methods of the present disclosure comprise heating a treatment sample. A “treatment sample” as referred to herein, is a sample (e.g., an aqueous solution or suspension) containing at least a nucleic acid and optionally reagents such as enzymes or chelating agents. In certain embodiments, a treatment sample may be a biological sample of nucleic acids collected from a subject and diluted with water or a buffer and optionally including the one or more reagents. The biological sample may be a bodily sample, such as saliva or mucus. The biological sample may comprise a cell (e.g., an unlysed cell). In certain embodiments, the sample of nucleic acid collected from a subject is isolated or purified, e.g., isolated or purified from the cell, proteins, or other biologics in the biological sample, prior to dilution and/or addition of reagents. In certain embodiments, the sample of nucleic acid collected from a subject is not isolated or purified, e.g., isolated or purified from the cell, proteins, or other biologics in the biological sample, prior to dilution and/or addition of reagents.

In some aspects, the method comprises heating the treatment sample to a temperature above 100 degrees Celsius in a closed heating chamber in hyperbaric heating conditions. For instance, the closed heating chamber may substantially prevent air and vapor from entering or leaving the chamber. In some cases, there is negligible air flow in and out of the closed heating chamber. The closed heating chamber may remain closed during hyperbaric heating. In some embodiments, “hyperbaric heating conditions” referred to herein are conditions in which heating generates a higher pressure insides the closed heating chamber than outside the closed heating chamber. In some embodiments, the temperature referred to herein is each an average temperature for the duration of the sample heating step or a portion thereof. In some embodiments, the temperature referred to herein is the temperature inside the closed vessel. In some embodiments, the temperature referred to herein is the temperatures of heating device used to hyperbarically heat the sample. In some cases, the temperature is estimated using an internal temperature sensor inside the closed vessel. In some cases, the internal temperature is correlated via an external temperature infrared sensor assessing the exterior temperature of the closed vessel. In some aspects, the heating of the treatment sample occurs at a temperature ramp rate, for example, from 6 degrees Celsius per second to 20 degrees Celsius per second. In some aspects, the methods of the present disclosure comprise heating the treatment sample from a first temperature to a second temperature above 100 degrees over a ramp time.

In some aspects, the methods of the present disclosure comprise analyzing a nucleic acid in a bodily sample selected from: blood, lacrimal fluid, saliva, mucus, sputum, feces, cerebrospinal fluid, and urine. In one aspect, the method comprises providing the bodily sample and heating a treatment sample comprising the bodily sample. In some embodiments, the nucleic acid is not extracted, isolated, or otherwise purified from the bodily sample.

In some aspects, the methods of the present disclosure comprise heating a treatment sample comprising an unlysed cell comprising a nucleic acid, thereby producing a heat-treated sample. In some embodiments, the method further comprises analyzing the nucleic acid. In some embodiments, the nucleic acid is not extracted, isolated, or otherwise purified from the heat-treated sample prior to analyzing the nucleic acid.

In some aspects, the treatment sample comprising the nucleic acid further comprises one or more reagents selected from: a chelating agent, a single stranded nucleic acid binding protein, and a reducing agent. In some embodiments, the treatment sample a chelating agent, a single stranded nucleic acid binding protein, and a reducing agent.

In some aspects, the present disclosure provides a method of inactivating a molecular amplification inhibitor in a treatment sample comprising a nucleic acid. In some aspects, the method comprises heating a treatment sample comprising i) a nucleic acid and ii) a plurality of molecular amplification inhibitors. In one aspect, the method comprises heating the treatment sample in a closed heating chamber to a temperature above 100 degrees Celsius in hyperbaric heating conditions, thereby inactivating a molecular amplification inhibitor of the plurality of amplification inhibitors and producing a heat-treated sample. In another aspect, the method comprises heating the treatment sample in a closed heating chamber in hyperbaric heating conditions from a first temperature to a second temperature over a ramp time above 100 degrees Celsius, thereby inactivating a molecular amplification inhibitor of said plurality of amplification inhibitors and producing a heat-treated sample, wherein the nucleic acid is not substantially degraded. In some embodiments, the method further comprises detecting the nucleic acid.

In some aspects, the methods described herein comprise heating a treatment sample in a closed heating chamber in hyperbaric conditions to a temperature above 100 degrees Celsius. In some embodiments, the method comprises hyperbaric heating the treatment sample to a temperature above 100 degrees Celsius. The temperature may be above the boiling point of water. In some embodiments, the treatment sample does not boil in the hyperbaric conditions at the temperature above 100 degrees. In some embodiments, the method comprises hyperbaric heating the treatment sample to a temperature from 100 degrees Celsius to 160 degrees Celsius. In some embodiments, the temperature is from 101 degrees Celsius to 160 degrees Celsius. In some embodiments, the temperature is from 105 degrees Celsius to 160 degrees Celsius. In some embodiments, the temperature is from 110 degrees Celsius to 160 degrees Celsius. In some embodiments, the temperature is from 120 degrees Celsius to 160 degrees Celsius. In some embodiments, the temperature is from 130 degrees Celsius to 160 degrees Celsius. In some embodiments, the temperature is from 140 degrees Celsius to 160 degrees Celsius. In some embodiments, the temperature is from 150 degrees Celsius to 160 degrees Celsius. In some embodiments, the temperature is from 100 degrees Celsius to 140 degrees Celsius. In some embodiments, the temperature is from 110 degrees Celsius to 140 degrees Celsius. In some embodiments, the temperature is from 120 degrees Celsius to 140 degrees Celsius. In some embodiments, the temperature is from 130 degrees Celsius to 140 degrees Celsius. In some embodiments, the temperature is about 110 degrees Celsius. In some embodiments, the temperature is about 120 degrees Celsius. In some embodiments, the temperature is about 130 degrees Celsius. In some embodiments, the temperature is about 140 degrees Celsius. In some embodiments, the temperature is about 150 degrees Celsius. In some embodiments, the temperature is about 160 degrees Celsius.

In some aspects, the heating of the treatment sample happens for a first time period. In some embodiments, the first time period is, for example, about 10 seconds, about 20 seconds, 30 seconds, 40 seconds, 50 seconds, 60 seconds, 70 seconds, 80 seconds, 90 seconds, 100 seconds, 110 seconds, 120 seconds, 130 seconds, 140 seconds, 150 seconds, 160 seconds, 170 seconds, 180 seconds, 190 seconds, 200 seconds or 300 seconds. In some embodiments, the first time period is, for example, from 1 second to 300 seconds, 5 seconds to 300 seconds, 30 seconds to 300 seconds, from 40 seconds to 300 seconds, from 50 seconds to 300 seconds, from 60 seconds to 300 seconds, from 70 seconds to 300 seconds, from 80 seconds to 300 seconds, from 90 seconds to 300 seconds, from 100 seconds to 300 seconds, from 110 seconds to 300 seconds, from 120 seconds to 300 seconds, or from 200 seconds to 300 seconds. In some embodiments, the first time period is, for example, from 10 seconds to 180 seconds, from 20 seconds to 180 seconds, from 30 seconds to 180 seconds, from 40 seconds to 180 seconds, from 50 seconds to 180 seconds, from 60 seconds to 180 seconds, from 70 seconds to 180 seconds, from 80 seconds to 180 seconds, from 90 seconds to 180 seconds, from 100 seconds to 180 seconds, from 110 seconds to 180 seconds, or from 120 seconds to 180 seconds. In some embodiments, the first time period is, for example, from 10 seconds to 120 seconds, from 20 seconds to 120 seconds, from 30 seconds to 120 seconds, from 40 seconds to 120 seconds, from 50 seconds to 120 seconds, from 60 seconds to 120 seconds, from 70 seconds to 120 seconds, from 80 seconds to 120 seconds, from 90 seconds to 120 seconds, from 100 seconds to 120 seconds, or from 110 seconds to 120 seconds. In some embodiments, the first time period is, for example, from 30 seconds to 90 seconds, from 40 seconds to 90 seconds, from 50 seconds to 90 seconds, from 60 seconds to 90 seconds, from 70 seconds to 90 seconds, or from 80 seconds to 90 seconds. In some embodiments, the first time period is, for example, from 10 seconds to 60 seconds, from 20 seconds to 60 seconds, 30 seconds to 60 seconds, from 40 seconds to 60 seconds, or from 50 seconds to 60 seconds.

In some embodiments, the first time period is, for example, about 1 minute, about 2 minutes, about 3 minutes, about 4 minutes, about 5 minutes, about 6 minutes, about 7 minutes, about 8 minutes, about 9 minutes, or about 10 minutes. In some embodiments, the first time period is, for example, from 1 minute to 2 minutes, from 1 minute to 3 minutes, from 1 minute to 4 minutes, from 1 minute to 5 minutes, from 1 minute to 6 minutes, from 1 minute to 7 minutes, from 1 minute to 8 minutes, from 1 minute to 9 minutes, or from 1 minute to 10 minutes. In some embodiments, the first time period is, for example, from 1 minute to 5 minutes, from 2 minutes to 5 minutes, from 3 minutes to 5 minutes, from 4 minutes to 5 minutes.

In some aspects, the method further comprises, after heating the treatment sample to a temperature above 100 degrees Celsius, maintaining the treatment sample at the temperature for a maintenance time prior to a cooling time. In some embodiments, the maintenance time is from 0 seconds to 300 seconds, from 0 seconds to 250 seconds, from 0 second to 200 seconds, from 0 seconds to 150 seconds, from 0 seconds to 120 seconds, from 0 seconds to 100 seconds, from 0 seconds to 50 seconds, from 0 seconds to 40 seconds, from 0 seconds to 30 seconds, from 0 seconds to 20 seconds, or from 1 to 10 seconds. In some embodiments, the maintenance time is from 5 seconds to 300 seconds, from 5 seconds to 250 seconds, from 5 seconds to 200 seconds, from 5 seconds to 150 seconds, from 5 seconds to 100 seconds, from 5 seconds to 50 seconds, from 5 seconds to 40 seconds, from 5 seconds to 30 seconds, from 5 seconds to 20 seconds, or from 5 to 10 seconds.

During the maintenance time, the closed heating chamber may remain closed (e.g., substantially prevent air and vapor from entering or leaving the chamber).

In some embodiments, the cooling time is from 5 seconds to 300 seconds. In some embodiments, the cooling time is from 30 seconds to 300 seconds. In some embodiments, the cooling time is from 60 seconds to 300 seconds. In some embodiments, the cooling time is from 90 seconds to 300 seconds. In some embodiments, the cooling time is from 120 seconds to 300 seconds. In some embodiments, the cooling time is from 150 seconds to 300 seconds. In some embodiments, the cooling time is from 180 seconds to 300 seconds. In some embodiments, the cooling time is from 240 seconds to 300 seconds. In some embodiments, the cooling time is from 270 seconds to 300 seconds.

In some embodiments, the cooling time is from 10 seconds to 120 seconds. In some embodiments, the cooling time is from 30 seconds to 120 seconds. In some embodiments, the cooling time is from 60 seconds to 120 seconds. In some embodiments, the cooling time is from 90 seconds to 120 seconds. During the cooling time, the closed heating chamber may remain closed (e.g., substantially prevent air and vapor from entering or leaving the chamber). In some embodiments, the nucleic acid is not substantially degraded after heating.

In some aspects, heating the treatment sample comprising said nucleic acid occurs at a temperature ramp rate. In some embodiments, the temperature ramp rate is calculated as the time derivative of temperature. In some embodiments, the temperature ramp rate is calculated as the average rate of change of temperature with respect to time. In some embodiments, the temperature ramp rate is at least 0.5 degrees Celsius per second, at least 1 degrees Celsius per second, at least 2 degrees Celsius per second, at least 3 degrees Celsius per second, at least 4 degrees Celsius per second, at least 5 degrees Celsius per second, at least 6 degrees Celsius per second, at least 7 degrees Celsius per second, at least 8 degrees Celsius per second, at least 9 degrees Celsius per second, at least 10 degrees Celsius per second, at least 11 degrees Celsius per second, at least 12 degrees Celsius per second, at least 13 degrees Celsius per second, at least 14 degrees Celsius per second, at least 15 degrees Celsius per second, at least 16 degrees Celsius per second, at least 17 degrees Celsius per second, at least 18 degrees Celsius per second, at least 19 degrees Celsius per second, at least 20 degrees Celsius per second, at least 25 degrees Celsius per second, at least 27.5 degrees Celsius per second, at least 30 degrees Celsius per second, at least 32.5 degrees Celsius per second at least 35 degrees Celsius per second, at least 37.5 degrees Celsius per second, at least 40 degrees Celsius per second, at least 42.5 degrees Celsius per second, at least 45 degrees Celsius per second, at least 47.5 degrees Celsius per second, or at least 50 degrees Celsius per second.

In some embodiments, the temperature ramp rate is from 0.5 degrees Celsius per second to 50 degrees Celsius per second, from 0.5 degrees Celsius per second to 40 degrees Celsius per second, from 0.5 degrees Celsius per second to 35 degrees Celsius per second, from 0.5 degrees Celsius per second to 30 degrees Celsius per second, from 0.5 degrees Celsius per second to 25 degrees Celsius per second, from 0.5 degrees Celsius per second to 20 degrees Celsius per second, or from 0.5 degrees Celsius per second to 15 degrees Celsius per second.

In some embodiments, the temperature ramp rate is from 2 degrees Celsius per second to 50 degrees Celsius per second, from 2 degrees Celsius per second to 40 degrees Celsius per second, from 2 degrees Celsius per second to 35 degrees Celsius per second, from 2 degrees Celsius per second to 30 degrees Celsius per second, from 2 degrees Celsius per second to 25 degrees Celsius per second, from 2 degrees Celsius per second to 20 degrees Celsius per second, or from 2 degrees Celsius per second to 15 degrees Celsius per second.

In some embodiments, the temperature ramp rate is from 5 degrees Celsius per second to 50 degrees Celsius per second, from 5 degrees Celsius per second to 40 degrees Celsius per second, from 5 degrees Celsius per second to 35 degrees Celsius per second, from 5 degrees Celsius per second to 30 degrees Celsius per second, from 5 degrees Celsius per second to 25 degrees Celsius per second, from 5 degrees Celsius per second to 20 degrees Celsius per second, or from 5 degrees Celsius per second to 15 degrees Celsius per second. In some embodiments, the nucleic acid is not substantially degraded after heating.

In some aspects, the methods described herein comprise heating a treatment sample comprising said nucleic acid in a closed heating chamber in hyperbaric conditions from a first temperature to a second temperature above 100 degrees Celsius over a ramp time, thereby producing a heat-treated sample. The closed heating chamber may remain closed (e.g., substantially preventing air from entering or exiting the chamber) during heating from the first temperature to the second temperature. In some embodiments, the treatment sample does not boil in the hyperbaric conditions at the second temperature above 100 degrees. In some embodiments, the first temperature is from 4 degrees Celsius to 40 degrees Celsius and the second temperature is from 100 degrees Celsius to 160 degrees Celsius. In some embodiments, the first temperature is from 4 degrees Celsius to 40 degrees Celsius and the second temperature is from 101 degrees Celsius to 160 degrees Celsius. In some embodiments, the first temperature is from 4 degrees Celsius to 40 degrees Celsius and the second temperature is from 105 degrees Celsius to 160 degrees Celsius. In some embodiments, the first temperature is from 4 degrees Celsius to 40 degrees Celsius and the second temperature is from 110 degrees Celsius to 160 degrees Celsius. In some embodiments, the first temperature is from 4 degrees Celsius to 40 degrees Celsius and the second temperature is from 120 degrees Celsius to 160 degrees Celsius. In some embodiments, the first temperature is from 4 degrees Celsius to 40 degrees Celsius and the second temperature is from 130 degrees Celsius to 160 degrees Celsius. In some embodiments, the first temperature is from 4 degrees Celsius to 40 degrees Celsius and the second temperature is from 140 degrees Celsius to 160 degrees Celsius.

In some embodiments, the first temperature is from 20 degrees Celsius to 30 degrees Celsius and the second temperature is from 100 degrees Celsius to 160 degrees Celsius. In some embodiments, the first temperature is from 20 degrees Celsius to 30 degrees Celsius and the second temperature is from 101 degrees Celsius to 160 degrees Celsius. In some embodiments, the first temperature is from 20 degrees Celsius to 30 degrees Celsius and the second temperature is from 105 degrees Celsius to 160 degrees Celsius. In some embodiments, the first temperature is from 20 degrees Celsius to 30 degrees Celsius and the second temperature is from 110 degrees Celsius to 160 degrees Celsius. In some embodiments, the first temperature is from 20 degrees Celsius to 30 degrees Celsius and the second temperature is from 120 degrees Celsius to 160 degrees Celsius. In some embodiments, the first temperature is from 20 degrees Celsius to 30 degrees Celsius and the second temperature is from 130 degrees Celsius to 160 degrees Celsius. In some embodiments, the first temperature is from 20 degrees Celsius to 30 degrees Celsius and the second temperature is from 140 degrees Celsius to 160 degrees Celsius.

In some embodiments, the first temperature is from 4 degrees Celsius and 40 degrees Celsius and the second temperature and the second temperature is at least 100 degrees Celsius. In some embodiments, the first temperature is from 4 degrees Celsius and 40 degrees Celsius and the second temperature and the second temperature is at least 101 degrees Celsius. In some embodiments, the first temperature is from 4 degrees Celsius and 40 degrees Celsius and the second temperature and the second temperature is at least 105 degrees Celsius. In some embodiments, the first temperature is from 4 degrees Celsius and 40 degrees Celsius and the second temperature and the second temperature is at least 110 degrees Celsius. In some embodiments, the first temperature is from 4 degrees Celsius and 40 degrees Celsius and the second temperature and the second temperature is at least 120 degrees Celsius. In some embodiments, the first temperature is from 4 degrees Celsius and 40 degrees Celsius and the second temperature and the second temperature is at least 130 degrees Celsius. In some embodiments, the first temperature is from 4 degrees Celsius and 40 degrees Celsius and the second temperature and the second temperature is at least 140 degrees Celsius. In some embodiments, the first temperature is from 4 degrees Celsius and 40 degrees Celsius and the second temperature and the second temperature is at least 150 degrees Celsius. In some embodiments, the first temperature is from 4 degrees Celsius and 40 degrees Celsius and the second temperature and the second temperature is at least 160 degrees Celsius.

In some embodiments, the ramp time is from 3 to 100 seconds, from 6 to 100 seconds, from 7 to 100 seconds, from 8 to 100 seconds, from 9 to 100 seconds, or from 10 to 100 seconds. In some embodiments, the ramp time is from 3 to 50 seconds, from 6 to 50 seconds, from 7 to 50 seconds, from 8 to 50 seconds, from 9 to 50 seconds, or from 10 to 50 seconds.

In some embodiments, the method of analyzing the nucleic acid further comprises, after (a), maintaining said treatment sample at the second temperature for a maintenance time prior to a cooling time. In some embodiments, the maintenance time is from 0 seconds to 300 seconds. In some embodiments, the maintenance time is from 5 seconds to 300 seconds. In some embodiments, the maintenance time is from 30 seconds to 300 seconds. In some embodiments, the maintenance time is from 60 seconds to 300 seconds. In some embodiments, the maintenance time is from 90 seconds to 300 seconds. In some embodiments, the maintenance time is from 120 seconds to 300 seconds. In some embodiments, the maintenance time is from 150 seconds to 300 seconds. In some embodiments, the maintenance time is from 180 seconds to 300 seconds. In some embodiments, the maintenance time is from 240 seconds to 300 seconds. In some embodiments, the maintenance time is from 270 seconds to 300 seconds.

In some embodiments, the maintenance time is from 0 seconds to 120 seconds. In some embodiments, the maintenance time is from 10 seconds to 120 seconds. In some embodiments, the maintenance time is from 30 seconds to 120 seconds. In some embodiments, the maintenance time is from 60 seconds to 120 seconds. In some embodiments, the maintenance time is from 90 seconds to 120 seconds. During the maintenance time, the closed heating chamber may remain closed (e.g., substantially prevent air and vapor from entering or leaving the chamber).

In some embodiments, the cooling time is from 0 seconds to 300 seconds. In some embodiments, the cooling time is from 5 seconds to 300 seconds. In some embodiments, the cooling time is from 30 seconds to 300 seconds. In some embodiments, the cooling time is from 60 seconds to 300 seconds. In some embodiments, the cooling time is from 90 seconds to 300 seconds. In some embodiments, the cooling time is from 120 seconds to 300 seconds. In some embodiments, the cooling time is from 150 seconds to 300 seconds. In some embodiments, the cooling time is from 180 seconds to 300 seconds. In some embodiments, the cooling time is from 240 seconds to 300 seconds. In some embodiments, the cooling time is from 270 seconds to 300 seconds.

In some embodiments, the cooling time is from 10 seconds to 120 seconds. In some embodiments, the cooling time is from 30 seconds to 120 seconds. In some embodiments, the cooling time is from 60 seconds to 120 seconds. In some embodiments, the cooling time is from 90 seconds to 120 seconds. During the cooling time, the closed heating chamber may remain closed (e.g., substantially prevent air and vapor from entering or leaving the chamber).

In some aspects, heating the treatment sample in a closed chamber generates a pressure inside the chamber. In some embodiments, the pressure inside of the chamber is from, e.g., 1 to 200 PSI, 10 to 200 PSI, 20 to 200 PSI, 30 to 200 PSI, 40 to 200 PSI, 50 to 200 PSI, 10 to 100 PSI, 20 to 100 PSI, 30 to 100 PSI, 40 to 100 PSI, 50 to 100 PSI, 60 to 100 PSI, 70 to 100 PSI, 80 to 100 PSI, or 90 to 100 PSI, each of which is over 1 atm. In some embodiments, the pressure inside of the chamber is from, e.g., 10 to 100 PSI, 10 to 90 PSI, 10 to 80 PSI, 10 to 70 PSI, 10 to 60 PSI, 10 to 50 PSI, 10 to 40 PSI, 10 to 30 PSI, 10 to 20 PSI, 20 to 100 PSI, 20 to 90 PSI, 20 to 80 PSI, 20 to 70 PSI, 20 to 60 PSI, 20 to 50 PSI, 20 to 40 PSI, 20 to 30 PSI, 30 to 100 PSI, 30 to 90 PSI, 30 to 80 PSI, 30 to 70 PSI, 30 to 60 PSI, 30 to 50 PSI, or 30 to 40 PSI, each of which is over 1 atm. In some embodiments, the pressure inside the chamber is from 40 to 60 PSI over 1 atm. In some embodiments, the pressure inside the chamber is from 40 to 50 PSI over 1 atm. In some embodiments, the nucleic acid is not substantially degraded after heating

Any means for heating the sample can be used, including but not limited to, contact heating in a dry heat block, induction heating, microwave heating, nanophotonic heating, or any other heating means known to the art. In some aspects, the treatment sample is heated with a heat source. In some embodiments, the heat source comprises an induction heater, a heating element, or a microwave. For example, in some embodiments, a heat block or heat bath can be used, e.g., by filling with pre-heated aluminum beads. In some embodiments, the heating of the biological sample is hyperbaric heating. In some embodiments, the treatment sample is hyperbarically heated with a heat source.

For example, in some embodiments, the treatment sample is heated using a heat block or heat bath is pre-heated at temperature ranging from 100 degrees Celsius to 300 degrees Celsius, or more preferably from 100 to 150 degrees Celsius where the hyperbaric heating vessel is placed on and leave for, e.g., 1 second to 10 minutes after reaching 100 degrees Celsius. In another embodiments, the vessel is hyperbarically heated for 20 to 40 seconds after reaching the temperature of 100 degrees Celsius or higher.

In another embodiment of the present disclosure, heat can be applied to the sample via induction heating. In some embodiments, the vessel can be made from a paramagnetic material or paramagnetic material can be placed directly in contact with the said sample inside the heating vessel made of non-paramagnetic heat resistant material such as plastic polymer. This provides an extreme temperature ramp rate enabling to bring the sample above 100 degrees Celsius in less than 30 seconds, which allow a complete sample preparation in one minute or less.

In some embodiments, a laser can be used to heat the treatment sample inside the vessel at temperature above 100 degrees Celsius (e.g., hyperbaric heating). Nanoparticles can be added to the heating system to capture laser energy and transduce it directly into the sample. This technic provides very high heating ramp rate compatible with superheating sample preparation. In some embodiments, the heat source is a laser. In some embodiments, nanoparticles are added to the laser.

In some aspects, the present disclosure provides methods of heating a treatment sample comprising a nucleic acid. In some embodiments, the nucleic acid is DNA. In some embodiments, the nucleic acid is RNA. In some embodiments, the nucleic acid is derived from a eukaryotic cell or a prokaryotic cell. In some embodiments, the nucleic acid is derived from a virus. In some embodiments, the nucleic acid is selected from a viral nucleic acid, a bacterial nucleic acid, a protozoan nucleic acid, a eukaryotic nucleic acid, and a fungal nucleic acid. In some embodiments, the nucleic acid is a viral nucleic acid.

In some embodiments, the treatment sample comprising a bodily sample comprising the nucleic acid is heated in a closed heating chamber in hyperbaric conditions. In some embodiments, the bodily sample comprises a substance selected from blood, plasma, serum, lacrimal fluid, saliva, mucus, sputum, feces, cerebrospinal fluid, lymph fluid, bile, synovial fluid, cyst fluid, ascites, pleural fluid, ocular fluid, interstitial fluid, cervical fluid, and urine. In some embodiments, the bodily sample is selected from blood, lacrimal fluid, saliva, mucus, sputum, feces, cerebrospinal fluid, and urine. In some embodiments, the bodily sample comprises mucus. In some embodiments, the bodily sample comprises body fluid sample, tissue, or cell of a subject. In some embodiments, the nucleic acid is not extracted, isolated, or otherwise purified from the bodily sample.

In some aspects, the bodily sample may be collected from a subject (e.g., a human). The bodily sample may be collected via nasopharyngeal swab, cervical swab, or nasal swab from said subject. In some cases, the bodily sample may comprise a pathogen or a portion thereof. In some embodiments, the pathogen or portion thereof is selected from a virus or a portion thereof, a bacterium or a portion thereof, a protozoon or a portion thereof, a yeast or a portion thereof, and a fungus or a portion thereof. In some embodiments, the pathogen is a blood-borne pathogen or portion thereof. In some embodiments, the pathogen is a respiratory pathogen or portion thereof.

In some embodiments, the treatment sample comprising a bodily sample comprising the nucleic acid is heated in a closed heating chamber in hyperbaric conditions to a temperature above 100 degrees Celsius, thereby producing a heat-treated sample. In some aspects, the nucleic acid is analyzed after hyperbaric heating. In some cases, the nucleic acid is not extracted, isolated, or otherwise purified from said bodily sample. For example, in some cases, the nucleic acid is not extracted by phenol chloroform extraction or purified via a commercial nucleic acid purification kit or purified via column chromatography.

In some aspects, a treatment sample comprising a cell comprising the nucleic acid is heated in a closed heating chamber in hyperbaric conditions. The cell may be an unlysed cell. In some embodiments, the cell is embedded in a biological matrix such as nasal mucus, cerebrospinal fluid, feces, vaginal mucus, urine, or saliva. In some embodiments, the cell is embedded in nasal mucus. In some embodiments, the cell is a bacteria cell, e.g., B. subtilis, E. Coli, or S. Pyogenes. In some embodiments, the cell is a fungal cell, e.g., C. albicans. In some embodiments, the cell is a yeast cell, e.g., S. cerevisiae. In some embodiments, the cell is a mammalian cell, e.g., a Chinese hamster ovary cell, a BHK cell, or a murine C127 cell. In some embodiments, the cell is a human cell, e.g., a HeLa cell. In some embodiments, the treatment sample comprises a bacterial spore, e.g., B. cereus bacterial spore.

In some embodiments, the treatment sample comprising the unlysed cell comprising a nucleic acid is heated in a closed heating chamber in hyperbaric conditions to a temperature above 100 degrees Celsius, thereby producing a heat-treated sample. In some embodiments, the nucleic acid is analyzed after hyperbaric heating. In some cases, the nucleic acid is not extracted, isolated, or otherwise purified from the heat-treated sample prior to analyzing the nucleic acid.

In some aspects, the present disclosure provides a method of inactivating a molecular amplification inhibitor in a treatment sample comprising the nucleic acid. In some cases, the molecular amplification inhibitor may be an agent that binds to nucleic acid. The molecular amplification inhibitor may be an agent that degrades nucleic acid. In some cases, the molecular amplification inhibitor may be a nuclease. In some cases, the molecular amplification inhibitor is a DNase. In other cases, the molecular amplification inhibitor is an RNase.

In some aspects, the method comprises heating a treatment sample comprising i) a nucleic acid and ii) a plurality of molecular amplification inhibitors. In one aspect, the method comprises heating the treatment sample in a closed heating chamber to a temperature above 100 degrees Celsius in hyperbaric heating conditions, thereby inactivating a molecular amplification inhibitor of the plurality of amplification inhibitors and producing a heat-treated sample. In another aspect, the method comprises heating the treatment sample in a closed heating chamber in hyperbaric heating conditions from a first temperature to a second temperature over a ramp time above 100 degrees Celsius, thereby inactivating a molecular amplification inhibitor of said plurality of amplification inhibitors and producing a heat-treated sample, wherein the nucleic acid is not substantially degraded. In some embodiments, the nucleic acid is not more than, e.g., 20%, 15%, 12%, 11%, 10%, 9%, 8%, 7%, 6%, 5%, 4%, 3%, 2%, or 1% degraded. In some embodiments, the method further comprises amplifying the nucleic acid using molecular amplification. In some embodiments, the method further comprises detecting the nucleic acid.

In some aspects, the method inactivates at least one molecular amplification inhibitor in the treatment sample. In some embodiments, the method inactivates at least 60% of the plurality of molecular amplification inhibitors. In some embodiments, the method inactivates at least 70% of the plurality of molecular amplification inhibitors. In some embodiments, the method inactivates at least 80% of the plurality of molecular amplification inhibitors. In some embodiments, the method inactivates at least 90% of the plurality of molecular amplification inhibitors.

In some aspects, the nucleic acid is not substantially degraded after heating, such as hyperbaric heating. In some embodiments, the nucleic acid is, e.g., not more than 20%, not more than 15%, not more than 12%, not more than 11%, not more than 10%, not more than 9%, not more than 8%, not more than 7%, not more than 6%, not more than 5%, not more than 4%, not more than 3%, not more than 2%, or not more than 1% degraded after hyperbaric heating. In some embodiments, the nucleic acid is, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, at least 99.5% intact after hyperbaric heating.

In some aspects, the nucleic acid is analyzed after hyperbaric heating. In some aspects, the method of analyzing the nucleic acid comprises amplifying the nucleic acid by molecular amplification (e.g., by a polymerase) after hyperbaric heating using any of the methods disclosed elsewhere herein. The molecular amplification may occur during real-time polymerase chain reaction (real-time PCR), transcription-mediated amplification (TMA), or loop-mediated isothermal amplification (LAMP). The molecular amplification of the nucleic acid may produce a molecular amplification product. In some embodiments, the method of analyzing the nucleic acid further comprises amplifying said nucleic acid by polymerase chain reaction (PCR) after heating, thereby producing a PCR product. In some embodiments, the method further comprises detecting the PCR product. In some cases, the PCR product is detected via a fluorescence signal emitted during amplification.

In some embodiments, the molecular amplification product (e.g., PCR product) is detectable following a lower number of molecular amplification cycles than would be required in the absence of hyperbaric heating of the treatment sample. In some embodiments, the molecular amplification product (e.g., PCR product) is detectable following a lower number of molecular amplification cycles than would be required as compared to heating the treatment sample at a temperature below 100 degrees Celsius. In some embodiments, the nucleic acid is detectable following a lower number of molecular amplification cycles than would be required as compared to heating the sample at a temperature below the boiling point of the sample for the same time.

In some embodiments, the molecular amplification product (e.g., PCR product) is detectable following from 10 to 50 molecular amplification cycles, from 15 to 50 molecular amplification cycles, from 20 to 50 molecular amplification cycles, from 25 to 50 molecular amplification cycles, from 26 to 50 molecular amplification cycles, from 27 to 50 molecular amplification cycles, from 28 to 50 molecular amplification cycles, from 29 to 50 molecular amplification cycles, or from 30 to 50 molecular amplification cycles.

In some embodiments, the molecular amplification product (e.g., PCR product) is detectable following from 10 to 40 molecular amplification cycles, from 20 to 40 molecular amplification cycles, from 25 to 40 molecular amplification cycles, from 26 to 40 molecular amplification cycles, from 27 to 40 molecular amplification cycles, from 28 to 40 molecular amplification cycles, from 29 to 40 molecular amplification cycles, or from 30 to 40 molecular amplification cycles.

In some embodiments, the molecular amplification product (e.g., PCR product) is detectable following from 10 to 35 molecular amplification cycles, from 20 to 35 molecular amplification cycles, from 25 to 35 molecular amplification cycles, from 26 to 35 molecular amplification cycles, from 27 to 35 molecular amplification cycles, from 28 to 35 molecular amplification cycles, from 29 to 35 molecular amplification cycles, or from 30 to 35 molecular amplification cycles. In some embodiments, the molecular amplification product (e.g., PCR product) is detectable following from 28 to 35 molecular amplification cycles. In some embodiments, the nucleic acid is detectable following, for example, from 25 to 35 molecular amplification cycles, from 25 to 34 molecular amplification cycles, from 25 to 33 molecular amplification cycles, from 25 to 32 molecular amplification cycles, from 25 to 31 molecular amplification cycles, from 25 to 30 molecular amplification cycles, from 25 to 29 molecular amplification cycles, or from 25 to 28 molecular amplification cycles.

In some embodiments, the molecular amplification product (e.g., PCR product) is detectable following 25 or more molecular amplification cycles. In some embodiments, the nucleic acid is detectable following, for example, 25 or more molecular amplification cycles, 26 or more molecular amplification cycles, 27 or more molecular amplification cycles, 28 or more molecular amplification cycles, 29 or more molecular amplification cycles, 30 or more molecular amplification cycles, 31 or more molecular amplification cycles, 32 or more molecular amplification cycles, 33 or more molecular amplification cycles, 34 or more molecular amplification cycles, or 35 or more molecular amplification cycles.

In some embodiments, the molecular amplification product (e.g., PCR product) is detected after the nucleic acid is amplified using from 10 to 40 molecular amplification cycles, from 10 to 35 molecular amplification cycles, from 20 to 35 molecular amplification cycles, from 25 to 35 molecular amplification cycles, from 26 to 35 molecular amplification cycles, from 27 to 35 molecular amplification cycles, from 28 to 35 molecular amplification cycles, from 29 to 35 molecular amplification cycles, or from 30 to 35 molecular amplification cycles.

In some embodiments, the detecting of the nucleic acid occurs simultaneously with the molecular amplification of the nucleic acid. In some embodiments, the detecting of the nucleic acid and the molecular amplification of the nucleic acid occur sequentially. In some embodiments, the detecting of the nucleic acid occurs after each cycle of molecular amplification.

In some embodiments, the method of analyzing the nucleic acid further comprises at least one of the following: detecting said nucleic acid, sequencing said nucleic acid, and genotyping said nucleic acid. In some embodiments, the method of analyzing the nucleic acid further comprises at least two of the following: detecting said nucleic acid, sequencing said nucleic acid, and genotyping said nucleic acid. In some embodiments, the method of analyzing the nucleic acid further comprises: detecting said nucleic acid, sequencing said nucleic acid, and genotyping said nucleic acid. In some embodiments, the method of analyzing the nucleic acid comprises detecting the nucleic acid via fluorescence detection.

As described herein, the treatment sample that is heated contains at least a nucleic acid and optionally reagents such as enzymes or chelating agents. In some embodiments, the nucleic acid is DNA. In some embodiments, the nucleic acid is RNA. In some embodiments, the nucleic acid is derived from a eukaryotic cell or a prokaryotic cell. In some embodiments, the nucleic acid is derived from a virus. In some embodiments, the nucleic acid is selected from a viral nucleic acid, a bacterial nucleic acid, a protozoan nucleic acid, a eukaryotic nucleic acid, and a fungal nucleic acid. In some embodiments, the nucleic acid is a viral nucleic acid.

In some embodiments, the nucleic acid is extracted from an organism selected from a prokaryote. In some embodiments, the nucleic acid is extracted from an organism selected from a eukaryote. In some embodiments, the nucleic acid is extracted from a parasite. In some embodiments, the nucleic acid is extracted from a virus, a bacterium, a fungus, an animal, or a plant.

In some embodiments, the nucleic acid is extracted from a virus. In some embodiments, the nucleic acid is extracted from a respiratory virus. In some embodiments, the virus is selected from an influenza virus, a rhinovirus, a coronavirus, a metapneumovirus, an adenoviruses, a syncytial virus, a bocaviruses, and a parainfluenza virus.

In some embodiments, the nucleic acid is extracted from a bacterium. In some embodiments, the nucleic acid is extracted from a gram-negative bacterium. In some embodiments, the nucleic acid is extracted from a gram-positive bacterium. In some embodiments, the nucleic acid is extracted from a fungus. In some embodiments, the nucleic acid is extracted from a yeast. In some embodiments, the nucleic acid is extracted from an animal. In some embodiments, the nucleic acid is extracted from a plant.

In some cases, the nucleic acid is in a bodily sample. In some embodiments, the nucleic acid is not extracted, isolated, or otherwise purified from the bodily sample. The bodily sample may be selected from blood, lacrimal fluid, saliva, mucus, sputum, feces, cerebrospinal fluid, and urine. In some embodiments, the bodily sample comprises a substance selected from blood, plasma, serum, lacrimal fluid, saliva, mucus, sputum, feces, cerebrospinal fluid, lymph fluid, bile, synovial fluid, cyst fluid, ascites, pleural fluid, ocular fluid, interstitial fluid, cervical fluid, and urine. In some embodiments, the bodily sample comprises mucus. In some embodiments, the bodily sample comprises body fluid sample, tissue, or cell of a subject.

In some aspects, the bodily sample may be collected from a subject. In some embodiments, the subject is a mammal. In some embodiments, the mammal is non-human primates (such as marmosets, macaques, chimpanzees), rodents (such as mouses, rats, gerbil jird, globefish Mouses, hamsters, cotton mouses, naked moles), rabbits, livestock mammals (such as goats, sheep, pigs, milk cows, ox, horses, camels), pet animals (such as dogs, cats), or zoo mammals. In some embodiments, the subject is a human.

The bodily sample may be collected via nasopharyngeal swab, cervical swab, or nasal swab from said subject. In some cases, the bodily sample may comprise a pathogen or a portion thereof. In some embodiments, the pathogen or portion thereof is selected from a virus or a portion thereof, a bacterium or a portion thereof, a protozoon or a portion thereof, a yeast or a portion thereof, and a fungus or a portion thereof. In some embodiments, the pathogen is a blood-bome pathogen or portion thereof. In some embodiments, the pathogen is a respiratory pathogen or portion thereof. In some embodiments, the respiratory pathogen comprises bacterial or fungal pathogens. In some embodiments, the respiratory pathogen is Streptococcus pneumoniae, Haemophilus influenzae, Moraxella catarrhalis, Streptococcus pyogenes, Chlamydophila pneumoniae, Mycoplasma pneumoniae, Bordetella pertussis, Klebsiella pneumoniae, Staphylococcus aureus, or Aspergillus sp. In some embodiments, the pathogen is a virus or a portion thereof. In some embodiments, the virus is respiratory virus. In some embodiments, the virus is SARS-CoV2, Influenza A viruses (-H1N1 and other subtypes), Influenza B virus, Human Respiratory Syncycial Virus (HRSV), Human Parainfluenza Viruses Type I (HPIV-1), II (HPIV-2), III (HPIV-3), IV (HPIV-4), Rhinovirus/Enterovirus (RV/EV), Adenoviruses (ADVs), Human Metapneumovirus (hMPV), Human Coronavirus (HCoV)-229E, HCoV-HKU1, HCoV-NL63, or HCoV-OC43. In some embodiments, the virus is SARS-CoV2.

In some embodiments, the virus is selected from an influenza virus, a rhinovirus, a coronavirus, a metapneumovirus, an adenoviruses, a syncytial virus, a bocaviruses, and a parainfluenza virus. In some embodiments, the virus is an influenza virus. In some embodiments, the virus is a rhinovirus. In some embodiments, the virus is a coronavirus. In some embodiments, the virus is a metapneumovirus. In some embodiments, the virus is an adenoviruses. In some embodiments, the virus is a syncytial virus. In some embodiments, the virus is a bocaviruses. In some embodiments, the virus is a parainfluenza virus.

In some aspects, the treatment sample comprises the bodily sample and is heated in a closed heating chamber in hyperbaric conditions to a temperature above 100 degrees Celsius, thereby producing a heat-treated sample.

In some aspects, the treatment sample comprises a cell comprising the nucleic acid. The cell may be an unlysed cell. The cell may be a eukaryotic cell or a prokaryotic cell. In some embodiments, the cell is a bacteria cell, e.g., B. subtilis, E. Coli, or S. Pyogenes. In some embodiments, the cell is a fungal cell, e.g., C. albicans. In some embodiments, the cell is a yeast cell, e.g., S. cerevisiae. In some embodiments, the cell is a mammalian cell, e.g., a Chinese hamster ovary cell, a BHK cell, or a murine C127 cell. In some embodiments, the cell is a human cell, e.g., a HeLa cell. In some embodiments, the cell is a bacterial spore, e.g., B. cereus bacterial spore.

In some embodiments, the cell may be in a bodily sample. In some embodiments, the cell is embedded in a biological matrix such as nasal mucus, cerebrospinal fluid, feces, vaginal mucus, urine, or saliva. In some embodiments, the cell is embedded in nasal mucus.

The treatment sample may be an aqueous solution or suspension. In some embodiments, the treatment sample comprises isolated nucleic acid diluted in a collection buffer. In other embodiments, the treatment sample comprises a bodily sample (e.g., saliva or mucus) comprising a nucleic acid that is diluted in a collection buffer. In further embodiments, the treatment sample comprises an undiluted bodily sample comprising the nucleic acid. In some embodiments, the treatment sample comprises a cell that is suspended in a collection buffer. The collection solution or buffer in the present disclosure can be made of pure water, or can be a mix of a low buffer capacity buffer. An example of such buffer could be based on Tris HCl buffer ranging from 1 mM to 50 mM, with or without EDTA at a concentration ranging from 0.5 mM to 1 mM.

In some embodiments, the treatment sample comprises a pH of from about 8.0 to about 12.0. In some embodiments, the treatment sample comprises a pH of from about 9.0 to about 12.0. In some embodiments, the treatment sample comprises a pH of from about 10.0 to about 12.0. In some embodiments, the treatment sample comprises a pH of from about 11.0 to about 12.0. In some embodiments, the treatment sample comprises a pH of from about 8.0 to about 11.0. In some embodiments, the treatment sample comprises a pH of from about 9.0 to about 11.0. In some embodiments, the treatment sample comprises a pH of from about 10.0 to about 11.0. In some embodiments, the treatment sample comprises a pH of from about 8.0 to about 10.0. In some embodiments, the treatment sample comprises a pH of from about 9.0 to about 10.0.

In some embodiments, the treatment sample comprises a pH above 7.0. In some embodiments, the treatment sample comprises a pH above 8.0. In some embodiments, the treatment sample comprises a pH above 8.5. In some embodiments, the treatment sample comprises a pH above 9.0. In some embodiments, the treatment sample comprises a pH above 9.5. In some embodiments, the treatment sample comprises a pH above 10.0. In some embodiments, the treatment sample comprises a pH above 10.5. In some embodiments, the treatment sample comprises a pH above 11.0.

In some embodiments, the treatment sample comprises a pH about 7.0. In some embodiments, the treatment sample comprises a pH about 8.0. In some embodiments, the treatment sample comprises a pH about 9.0. In some embodiments, the treatment sample comprises a pH about 10.0. In some embodiments, the treatment sample comprises a pH about 11.0. In some embodiments, the treatment sample comprises a pH about 12.0.

In some embodiments, the treatment sample comprises a pH of from about 8.0 to 11.0. In some embodiments, the treatment sample comprises a pH of from about 9.0 to 11.0. In some embodiments, the treatment sample comprises a pH of from about 10.0 to 11.0.

In some embodiments, the treatment sample comprises a pH of from about 8.0 to 12.0. In some embodiments, the treatment sample comprises a pH of from about 9.0 to 12.0. In some embodiments, the treatment sample comprises a pH of from about 10.0 to 12.0. In some embodiments, the treatment sample comprises a pH of from about 11.0 to 12.0.

In some embodiments, the treatment sample comprises a pH of from about 4.0 to about 7.0. In some embodiments, the treatment sample comprises a pH of from about 5.0 to about 7.0. In some embodiments, the treatment sample comprises a pH of from about 6.0 to about 7.0. In some embodiments, the treatment sample comprises a pH of from about 4.0 to about 6.0. In some embodiments, the treatment sample comprises a pH of from about 5.0 to about 6.0. In some embodiments, the treatment sample comprises a pH of from about 4.0 to about 5.0.

In some embodiments, the treatment sample comprises a pH about 3.0. In some embodiments, the treatment sample comprises a pH about 4.0. In some embodiments, the treatment sample comprises a pH about 5.0. In some embodiments, the treatment sample comprises a pH about 6.0. In some embodiments, the treatment sample comprises a pH about 7.0.

In some embodiments, the treatment sample comprises a pH below about 3.0. In some embodiments, the treatment sample comprises a pH below about 4.0. In some embodiments, the treatment sample comprises a pH below about 5.0. In some embodiments, the treatment sample comprises a pH below about 6.0. In some embodiments, the treatment sample comprises a pH a below bout 7.0.

In some embodiments, the volume of the treatment sample is the total volume of the mixture of the biological sample, the collection buffer, to one or more additives prior to hyperbaric heating. In some embodiments, the volume of the treatment sample is from 100 μL to 5 mL. In some embodiments, the volume of the treatment sample is from 200 μL to 5 mL. In some embodiments, the volume of the treatment sample is from 300 μL to 5 mL. In some embodiments, the volume of the treatment sample is from 400 μL to 5 mL. In some embodiments, the volume of the treatment sample is from 500 μL to 5 mL. In some embodiments, the volume of the treatment sample is from 1 mL to 5 mL. In some embodiments, the volume of the treatment sample is from 1.5 mL to 5 mL. In some embodiments, the volume of the treatment sample is from 2 mL to 5 mL. In some embodiments, the volume of the treatment sample is from 2.5 mL to 5 mL. In some embodiments, the volume of the treatment sample is from 3 mL to 5 mL. In some embodiments, the volume of the treatment sample is from 3.5 mL to 5 mL. In some embodiments, the volume of the treatment sample is from 4 mL to 5 mL. In some embodiments, the volume of the treatment sample is from 4.5 mL to 5 mL.

In some embodiments, the treatment sample comprises from 10 copies/mL to 10⁹ copies/mL of the nucleic acid. In some embodiments, the treatment sample comprises from 50 copies/mL to 10⁹ copies/mL of the nucleic acid. In some embodiments, the treatment sample comprises from 100 copies/mL to 10⁹ copies/mL of the nucleic acid. In some embodiments, the treatment sample comprises from 10³ copies/mL to 10⁹ copies/mL of the nucleic acid. In some embodiments, the treatment sample comprises from 10⁴ copies/mL to 10⁹ copies/mL of the nucleic acid. In some embodiments, the treatment sample comprises from 105 copies/mL to 10⁹ copies/mL of the nucleic acid. In some embodiments, the treatment sample comprises from 10⁶ copies/mL to 10⁹ copies/mL of the nucleic acid.

In some embodiments, the treatment sample comprises from 10 copies/mL to 10⁸ copies/mL of the nucleic acid. In some embodiments, the treatment sample comprises from 50 copies/mL to 10⁸ copies/mL of the nucleic acid. In some embodiments, the treatment sample comprises from 100 copies/mL to 10⁸ copies/mL of the nucleic acid. In some embodiments, the treatment sample comprises from 10³ copies/mL to 10⁸ copies/mL of the nucleic acid. In some embodiments, the treatment sample comprises from 10⁴ copies/mL to 10⁸ copies/mL of the nucleic acid. In some embodiments, the treatment sample comprises from 10⁵ copies/mL to 10⁸ copies/mL of the nucleic acid. In some embodiments, the treatment sample comprises from 10⁶ copies/mL to 10⁸ copies/mL of the nucleic acid.

In some embodiments, the treatment sample comprises from 10 copies/mL to 10⁷ copies/mL of the nucleic acid. In some embodiments, the treatment sample comprises from 50 copies/mL to 10⁷ copies/mL of the nucleic acid. In some embodiments, the treatment sample comprises from 100 copies/mL to 10⁷ copies/mL of the nucleic acid. In some embodiments, the treatment sample comprises from 10³ copies/mL to 10⁷ copies/mL of the nucleic acid. In some embodiments, the treatment sample comprises from 10⁴ copies/mL to 10⁷ copies/mL of the nucleic acid. In some embodiments, the treatment sample comprises from 10⁵ copies/mL to 10⁷ copies/mL of the nucleic acid. In some embodiments, the treatment sample comprises from 10⁶ copies/mL to 10⁷ copies/mL of the nucleic acid.

In some aspects, the treatment sample comprises one or more additives. The one or more additives may comprise a chelating agent, a single-stranded nucleic acid binding protein, a reducing agent, a protease, a nuclease inhibitor, or combination thereof.

In some embodiments, the treatment sample comprises a chelating agent. In some embodiments, the chelating agent is an insoluble chelating agent. In some embodiments, the insoluble chelating agent comprises a chelating resin. The chelating resin may be a polymer or copolymer. The chelating resin may be a cation-binding agent or a metal ion-binding agent. The chelating resin may be in the form of microbeads. In some embodiments, the chelating agent comprises crosslinked polystyrene. The chelating agent may comprise one or more functional groups. The one or more functional groups may comprise a sulfonic acid or sulfonate group; a quaternary amino group (e.g., trimethylammonium); a primary, secondary, and/or tertiary amino group (e.g., polyethylene amine); or a carboxylic acid or carboxylate group. In some embodiments, the insoluble chelating agent comprises a styrene divinylbenzene co-polymer. In some embodiments, the insoluble chelating agent comprises Chelex resin or Chelex. In some embodiments, the Chelex is stored in the collection buffer.

In some embodiments, the chelating agent is a soluble chelating agent. In some embodiments, the soluble chelating agent comprises ethylenediaminetetraacetic acid (EDTA). In some embodiments, the chelating agent comprises EDTA, nitrilotriacetic acid, n-hydroxyethylethylenediaminetriacetic acid (HEDTA), ethylenediamine, dimercaprol, porphine, heme, hemoglobin, or chlorophyll. In some embodiments, the chelating agent comprises simple organic acids such as oxalic acid, malic acid, rubeanic acid, or citric acid.

In some embodiments, the chelating agent is added at a final concentration, defined as the percent weight by volume of the chelating agent weight by the volume of the treatment sample (e.g., mixture comprising biological sample, collection buffer, chelating agent, single stranded nucleic acid binding protein, and reducing agent) prior to heating. In some embodiments, the final concentration of chelating agent in the treatment sample prior to heating is from 1% to 40%, from 2.5% to 35%, from 5% to 25%, from 7.5% to 20%, or from 10% to 15% weight by volume of the chelating agent weight by the treatment sample volume. In some embodiments, the final concentration of chelating agent in the treatment sample prior to heating is at about 2%, at about 4%, at about 6%, at about 8%, at about 10%, at about 12%, at about 14%, at about 16%, at about 18%, or at about 20% weight by volume of the chelating agent weight by the treatment sample volume.

In some embodiments, the treatment sample further comprises a single stranded nucleic acid binding (SSB) protein. In some embodiments, the single stranded nucleic acid binding protein is thermostable. In some embodiments, the SSB protein is thermostable at a temperature of from 4 degrees Celsius to 170 degrees Celsius, from 4 degrees Celsius to 160 degrees Celsius, from 4 degrees Celsius to 150 degrees Celsius, from 4 degrees Celsius to 140 degrees Celsius, from 4 degrees Celsius to 130 degrees Celsius, or from 4 degrees Celsius to 120 degrees Celsius. In some embodiments, the SSB protein is thermostable at a temperature of from 90 to 170 degrees Celsius, from 90 to 160 degrees Celsius, from 90 to 150 degrees Celsius, from 90 to 140 degrees Celsius, from 90 to 130 degrees Celsius, from 90 to 120 degrees Celsius, or from 90 to 110 degrees Celsius.

In some embodiments, the final molar concentration of the SSB protein in the treatment sample (e.g., mixture comprising biological sample, collection buffer, chelating agent, SSB protein, and reducing agent) prior to heating is at a concentration from 0.1 μM to 5 μM, from 0.1 μM to 4 μM, from 0.1 μM to 3 μM, from 0.1 μM to 2 μM, from 0.1 μM to 1 μM, from 0.2 μM to 0.9 μM, from 0.3 μM to 0.7 μM, or from 0.4 μM to 0.6 μM.

In some embodiments, the final molar concentration of the SSB protein in the treatment sample (e.g., mixture comprising biological sample, collection buffer, chelating agent, SSB protein, and reducing agent) prior to heating is at about 0.1 μM, at about 0.2 μM, at about 0.3 μM, at about 0.4 μM, at about 0.5 μM, at about 0.6 μM, at about 0.7 μM, at about 0.8 μM, at about 0.9 μM, at about 1 μM, at about 1.1 μM, at about 1.2 μM, at about 1.3 μM, at about 1.4 μM, at about 1.5 μM, at about 1.6 μM, at about 1.7 μM, at about 1.8 μM, at about 1.9 μM, at about 2 μM, at about 2.2 μM, at about 2.4 μM, 2.6 μM, at about 2.8 μM, at about 3 μM, at about 3.5 μM, at about 4 μM, at about 4.5 μM, or at about 5 μM.

In some embodiments, the single stranded nucleic acid binding protein is derived from a thermophilic organism. In some embodiments, the thermophilic organism is a thermophilic microorganism or a thermophilic bacteria. In some embodiments, the single stranded nucleic acid binding protein is derived from an organism selected from Thermotoga maritima (TmaSSB), Thermotoga neapolitana (TneSSB), Thermococcus kodakarensis (KOD), and Thermus thermophilus (TthSSB). In some embodiments, the single stranded nucleic acid binding protein is, for example, derived from Thermus aquaticus (TaqSSB) or Thermococcus kodakarensis (KOD). In some embodiments, the single stranded nucleic acid binding protein is derived from an organism selected from Thermotoga maritima (TmaSSB), Thermotoga neapolitana (TneSSB), and Thermus thermophilus (TthSSB). In some embodiments, the single stranded nucleic acid binding protein is derived from Thermotoga maritima (TmaSSB). In some embodiments, the single stranded nucleic acid binding protein is derived from Thermotoga neapolitana (TneSSB). In some embodiments, the single stranded nucleic acid binding protein is derived from Thermococcus kodakarensis (KOD). In some embodiments, the single stranded nucleic acid binding protein is derived from Thermus thermophilus (TthSSB). In some embodiments, the single stranded nucleic acid binding protein is selected from ET SSB, E. Coli SSB, KOD SSB, TthSSB, TneSSB, TmaSSB, and TaqSSB.

In some embodiments, the treatment sample comprises a reducing agent. In some embodiments the reducing agent is added to the treatment sample prior to heating. The reducing agent may be 2-Mercaptoethanol, 2-Mercaptoethylamine-HCl, TCEP, Cysteine-HCl, Dithiothreitol (DTT), TCEP-HCl, thiol-based reducing agent, Guanidine-HCl, or urea.

In some embodiments, the final molar concentration of the reducing agent in the treatment sample (e.g., mixture comprising biological sample, collection buffer, chelating agent, SSB protein, and reducing agent) prior to heating is at a concentration from 0.1 mM to 10 mM, 0.1 mM to 9 mM, from 0.1 mM to 8 mM, from 0.1 mM to 7 mM, from 0.1 mM to 6 mM, from 0.1 mM to 5 mM, from 0.1 mM to 4 mM, from 0.1 mM to 3 mM, from 0.1 mM to 2 mM, from 0.2 mM to 1.8 mM, from 0.4 mM to 1.6 mM, from 0.6 mM to 1.4 mM, or from 0.8 mM to 1.2 mM. In some embodiments, the final molar concentration of the reducing agent in the treatment sample (e.g., mixture comprising biological sample, collection buffer, chelating agent, SSB protein, and reducing agent) prior to heating is at about 0.1 mM, at about 0.2 mM, at about 0.3 mM, at about 0.4 mM, at about 0.5 mM, at about 0.6 mM, at about 0.7 mM, at about 0.8 mM, at about 0.9 mM, at about 1 mM, at about 1.2 mM, at about 1.4 mM, at about 1.6 mM, at about 1.8 mM, at about 2 mM, at about 2.5 mM, at about 5 mM, at about 7.5 mM, or at about 10 mM.

In some embodiments, the treatment sample comprises one or more reagents selected from: a chelating agent, a single stranded nucleic acid binding protein, and a reducing agent. In some embodiments, the treatment sample comprises at least two reagents selected from: a chelating agent, a single stranded nucleic acid binding protein, and a reducing agent. In some embodiments, the treatment sample comprises a chelating agent, a single stranded nucleic acid binding protein, and a reducing agent. In some embodiments, the concentration of the chelating agent is from 2.5% to 35% weight by volume of the treatment sample, the concentration of the single-stranded nucleic acid binding protein in the treatment sample is from 0.1 μM to 5 μM, and the concentration of the reducing agent in the treatment sample is from 0.1 mM to 5 mM. In some embodiments, the concentration of the chelating agent is from 5% to 25% weight by volume of the treatment sample, the concentration of the single-stranded nucleic acid binding protein in the treatment sample is from 0.1 μM to 2 μM, and the concentration of the reducing agent in the treatment sample is from 0.1 mM to 2 mM. In some embodiments, the concentration of the chelating agent is from 10% to 15% weight by volume of the treatment sample, the concentration of the single-stranded nucleic acid binding protein in the treatment sample is from 0.3 μM to 0.7 μM, and the concentration of the reducing agent in the treatment sample is from 0.6 mM to 1.4 mM.

In some embodiments, the treatment sample comprises a stabilizer. In some embodiments, the treatment sample is mixed with a stabilizer prior to heating. In some cases, the stabilizer may help prevent degradation of a nucleic acid. In some cases, the stabilizer may help inactivate a molecular amplification inhibitor during hyperbaric heating. In some embodiments, the stabilizer may stabilize a protein. In some embodiments, the stabilizer may stabilize a nucleic acid. In some embodiments, the stabilizer may stabilize a nucleic acid and a protein. A stabilizer may improve the viscosity of the treatment sample. A stabilizer may help prevent or reduce aggregation between one or more proteins. A stabilizer may improve solubility of a protein. A stabilizer may reduce nonspecific binding between one or more components (e.g., proteins). A stabilizer may be a blocking agent. A stabilizer may be a component that stabilizes one or more components in the treatment sample. In some embodiments, the stabilizer may stabilize the nucleic acid. In some embodiments, the stabilizer may stabilize the chelating agent. In some embodiments, the stabilizer may stabilize the single-stranded nucleic acid binding protein. For example, a stabilizer may be bovine serum albumin (BSA). A stabilizer may be gelatin. In some embodiments, the stabilizer has a concentration from 100 ng/mL to 15 mg/mL, from 200 ng/mL to 15 mg/mL, from 300 ng/mL to 15 mg/mL, from 400 ng/mL to 15 mg/mL, from 500 ng/mL to 15 mg/mL, or from 1 mg/mL to 15 mg/mL in the treatment sample. In some embodiments, the stabilizer has a concentration from 100 ng/mL to 10 mg/mL, from 200 ng/mL to 10 mg/mL, from 300 ng/mL to 10 mg/mL, from 400 ng/mL to 10 mg/mL, from 500 ng/mL to 10 mg/mL, or from 1 mg/mL to 10 mg/mL in the treatment sample. In some embodiments, the stabilizer has a concentration from 500 ng/mL to 2 mg/mL, from 500 ng/mL to 3 mg/mL, from 500 ng/mL to 4 mg/mL, from 500 ng/mL to 5 mg/mL, from 500 ng/mL to 6 mg/mL, from 500 ng/mL to 7 mg/mL, from 500 ng/mL to 8 mg/mL, from 500 ng/mL to 9 mg/mL, or from 500 ng/mL to 10 mg/mL in the treatment sample.

In some embodiments, the treatment sample comprises one or more reagents selected from: a chelating agent, a single stranded nucleic acid binding protein, a reducing agent, stabilizer. In some embodiments, the treatment sample comprises at least two reagents selected from: a chelating agent, a single stranded nucleic acid binding protein, a reducing agent, and a stabilizer. In some embodiments, the treatment sample comprises at least three reagents selected from: a chelating agent, a single stranded nucleic acid binding protein, a reducing agent, and a stabilizer. In some embodiments, the treatment sample comprises a chelating agent, a single stranded nucleic acid binding protein, and a reducing agent. In some embodiments, the concentration of the chelating agent is from 2.5% to 35% weight by volume of the treatment sample, the concentration of the single-stranded nucleic acid binding protein in the treatment sample is from 0.1 μM to 5 μM, the concentration of the reducing agent in the treatment sample is from 0.1 mM to 5 mM, and the concentration of the stabilizer is from 500 ng/mL to 10 mg/mL. In some embodiments, the concentration of the chelating agent is from 5% to 25% weight by volume of the treatment sample, the concentration of the single-stranded nucleic acid binding protein in the treatment sample is from 0.1 μM to 2 μM, the concentration of the reducing agent in the treatment sample is from 0.1 mM to 2 mM, and the concentration of the stabilizer is from 500 ng/mL to 10 mg/mL. In some embodiments, the concentration of the chelating agent is from 10% to 15% weight by volume of the treatment sample, the concentration of the single-stranded nucleic acid binding protein in the treatment sample is from 0.3 μM to 0.7 μM, the concentration of the reducing agent in the treatment sample is from 0.6 mM to 1.4 mM, and the concentration of the stabilizer is from 500 ng/mL to 10 mg/mL.

In some embodiments, the treatment sample comprises a protease. In some embodiments, the treatment sample is mixed with a protease prior to heating. In some embodiments, the protease is, for example, Proteinase K. In some embodiments, the biological sample is mixed with a reducing agent and a protease prior to heating. In some embodiments, the final concentration of the protease in the treatment sample (e.g., mixture comprising biological sample, collection buffer, chelating agent, SSB protein, reducing agent, and protease) prior to heating is at a concentration from 0.01 mg/mL to 5 mg/mL, from 0.02 mg/mL to 4 mg/mL, from 0.03 mg/mL to 3 mg/mL, from 0.04 mg/mL to 2 mg/mL, from 0.05 mg/mL to 1 mg/mL, from 0.075 mg/mL to 0.75 mg/mL, or from 0.1 mg/mL to 0.5 mg/mL. In some embodiments, the final concentration of the protease in the treatment sample (e.g., mixture comprising biological sample, collection buffer, chelating agent, SSB protein, reducing agent, and protease) prior to heating is at about 0.05 mg/mL, at about 0.075 mg/mL, at about 0.1 mg/mL, at about 0.25 mg/mL, at about 0.5 mg/mL, at about 0.75 mg/mL, or at about 1 mg/mL.

In some embodiments, the treatment sample comprises a nuclease inhibitor. In some embodiments, the treatment sample comprises an RNAse inhibitor. In some embodiments, an amount of RNAse inhibitor is added prior to heating, e.g., at about 1 U, at about 10 U, at about 50 U, at about 100 U, at about 150 U, at about 200 U, at about 250 U, at about 300 U, at about 350 U, at about 400 U, at about 450 U, or at about 500 U.

In some embodiments, an amount of RNAse inhibitor in the treatment sample is from about 1 U to about 500 U. In some embodiments, the amount of RNAse inhibitor in the treatment sample is from about 10 U to about 500 U. In some embodiments, the amount of RNAse inhibitor in the treatment sample is from about 50 U to about 500 U. In some embodiments, the amount of RNAse inhibitor in the treatment sample is from about 100 U to about 500 U. In some embodiments, the amount of RNAse inhibitor in the treatment sample is from about 150 U to about 500 U. In some embodiments, the amount of RNAse inhibitor in the treatment sample is from about 200 U to about 500 U. In some embodiments, the amount of RNAse inhibitor in the treatment sample is from about 250 U to about 500 U. In some embodiments, the amount of RNAse inhibitor in the treatment sample is from about 300 U to about 500 U. In some embodiments, the amount of RNAse inhibitor in the treatment sample is from about 350 U to about 500 U. In some embodiments, the amount of RNAse inhibitor in the treatment sample is from about 400 U to about 500 U. In some embodiments, the amount of RNAse inhibitor in the treatment sample is from about 450 U to about 500 U.

In some embodiments, the amount of RNAse inhibitor in the treatment sample is from about 1 U to about 200 U. In some embodiments, the amount of RNAse inhibitor in the treatment sample is from about 10 U to about 200 U. In some embodiments, the amount of RNAse inhibitor in the treatment sample is from about 50 U to about 200 U. In some embodiments, the amount of RNAse inhibitor in the treatment sample is from about 100 U to about 200 U. In some embodiments, the amount of RNAse inhibitor in the treatment sample is from about 150 U to about 200 U.

In some embodiments, the treatment sample further comprises a molecular amplification inhibitor. In some cases, the molecular amplification inhibitor may be an agent that binds to nucleic acid. The molecular amplification inhibitor may be an agent that degrades nucleic acid. In some cases, the molecular amplification inhibitor may be a nuclease. In some cases, the molecular amplification inhibitor is a DNase. In other cases, the molecular amplification inhibitor is an RNase. In some embodiments, the treatment sample comprises one or more molecular amplification inhibitors that may be inactivated after heating.

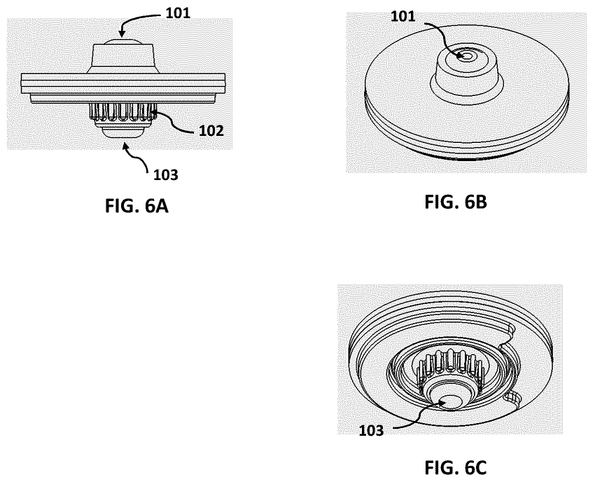

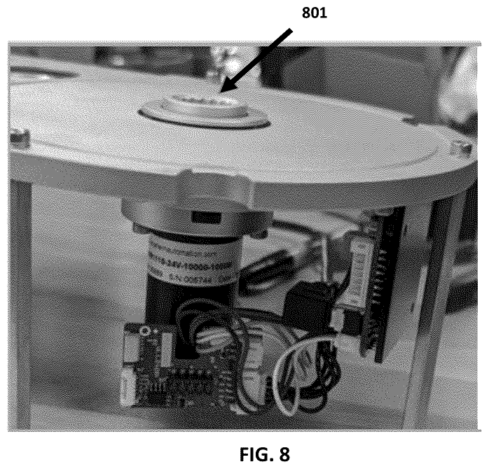

As described herein, a treatment sample may be heated in a closed heating chamber. A closed heating chamber may substantially prevent air and vapor from entering or leaving the chamber. In some cases, there is negligible air flow in and out of the closed heating chamber. The closed heating chamber may remain closed during hyperbaric heating. In some embodiments, the closed heating chamber is a chamber inside a heating vessel. The heating vessel and/or closed heating chamber may be composed of different materials. In some embodiments, the heating vessel and/or closed heating chamber comprises glass. In some embodiments, the heating vessel and/or closed heating chamber comprises high temperature polycarbonate. In some embodiments, the heating vessel and/or closed heating chamber comprises a thermally conductive material. In some embodiments, the heating vessel and/or closed heating chamber comprises metal. In some embodiments, the heating vessel and/or closed heating chamber may comprise zinc, stainless steel, copper, copper alloys, gold, silver, aluminum, aluminum nitride, iron, nickel, nickel allows, cobalt, carbon fiber, platinum, brass, tungsten, silicon, silicon carbide, diamond, or graphite. In some embodiments, the heating vessel and/or closed heating chamber comprises an electrically conductive material. In some embodiments, the heating vessel and/or closed heating chamber comprises a ferromagnetic material, such as iron, nickel, cobalt. For example, the heating vessel and/or closed heating chamber may be or comprise a glass ampule, a plastic container, or a metal container. In some embodiments, the heating vessel and/or closed heating chamber comprises an induction susceptor 103 (e.g., a metallic cup) (FIGS. 6, 7, 9) that can be used to heat the treatment sample by induction heating through contact with an induction platform 801 (FIG. 8).

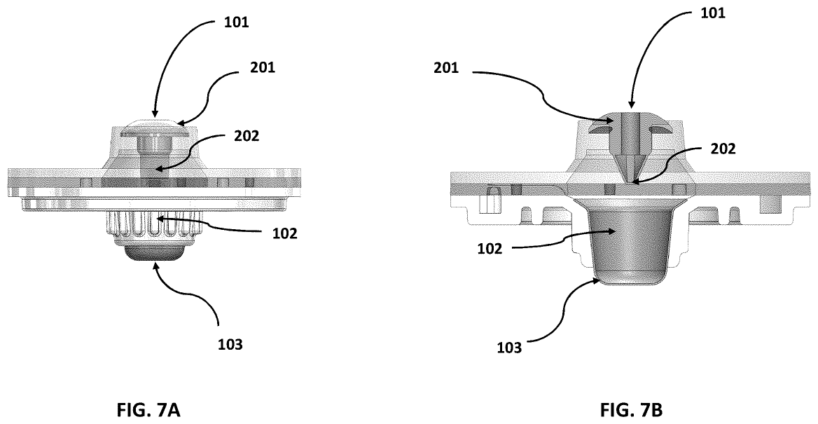

In some embodiments, the heating vessel comprises a heating chamber 702 that contains the treatment sample during hyperbaric heating (FIG. 7). The material of the vessel and/or heating chamber may be selected such that the heating chamber can have a temperature ramp rate of from 2 degrees Celsius per second to 50 degrees Celsius per second, from 2 degrees Celsius per second to 40 degrees Celsius per second, from 2 degrees Celsius per second to 35 degrees Celsius per second, from 2 degrees Celsius per second to 30 degrees Celsius per second, from 2 degrees Celsius per second to 25 degrees Celsius per second, from 2 degrees Celsius per second to 20 degrees Celsius per second, or from 2 degrees Celsius per second to 15 degrees Celsius per second. The material of the vessel and/or heating chamber may be selected such that the heating chamber can have a temperature ramp rate from 5 degrees Celsius per second to 60 degrees Celsius per second, from 5 degrees Celsius per second to 50 degrees Celsius per second, from 5 degrees Celsius per second to 40 degrees Celsius per second, from 5 degrees Celsius per second to 35 degrees Celsius per second, from 5 degrees Celsius per second to 30 degrees Celsius per second, from 5 degrees Celsius per second to 25 degrees Celsius per second, from 5 degrees Celsius per second to 20 degrees Celsius per second, or from 5 degrees Celsius per second to 15 degrees Celsius per second.

In some embodiments, the heating chamber is closed during hyperbaric heating and prevents air and vapor from entering or leaving the chamber. In some embodiments, the closed heating chamber remains closed in hyperbaric conditions, where the pressure inside the chamber is higher than the pressure outside the chamber. The material of the vessel or the material of the heating chamber may be selected such that the closed heating chamber can remain closed during hyperbaric heating, e.g., from room temperature to 160 degrees Celsius or when the pressure inside of the heating chamber is from 1 to 200 PSI over 1 atm.

In some embodiments, the closed heating chamber can be or comprise a glass ampule. The glass ampule may comprise a sample inlet that is fused. In other embodiments, the closed heating chamber can be or comprise a plastic container. The plastic container can comprise plastic resin or thermoresistant plastic comprising polycarbonate, high density polypropylene, PEEK, or PEI. For example, the plastic container may be a cryogenic tube or be part of a microfluidic cartridge. In further embodiments, the closed heating chamber can be or comprise a metal container. The metal container may comprise an induction susceptor (e.g., a metallic cup).

In some embodiments, the closed heating chamber is produced by sealing an open heating chamber. The sealing operation may comprise fusing an open sample inlet. The sealing operation may comprise using a cap, lid, plug, or valve. The manner of sealing the heating chamber may be selected such that the closed heating chamber may remain closed during hyperbaric heating.

In some embodiments, the heating vessel comprises a sample inlet 101 through which the sample may enter the heating chamber 102 as depicted in FIGS. 6, 7, 9, and 11. The sample inlet may be sealed to generate a closed heating chamber within the heating vessel. In some embodiments, the sample inlet is sealed using a cap, lid, plug, or valve. In some embodiments, the sample inlet may comprise a valve. The valve may be a one-way valve 201 (e.g., a duckbill valve) (FIG. 7A, 7B) that allows the sample to enter the heating chamber and prevents air and/or sample from exiting the heating chamber. In some cases, the sample is introduced through the one-way valve 201 (FIGS. 7A, 7B). The sample may be introduced manually using a pipette or injection needle. In other cases, the sample may be introduced through an automated system. The heating vessel may be part of a fluidics system and the one-way valve may be in contact with an upstream component of the fluidics system. In some embodiments, the one-way valve can further prevent air and vapor from exiting the heating chamber. The one-way valve provides a seal 202 for the closed chamber during hyperbaric heating (FIG. 7).

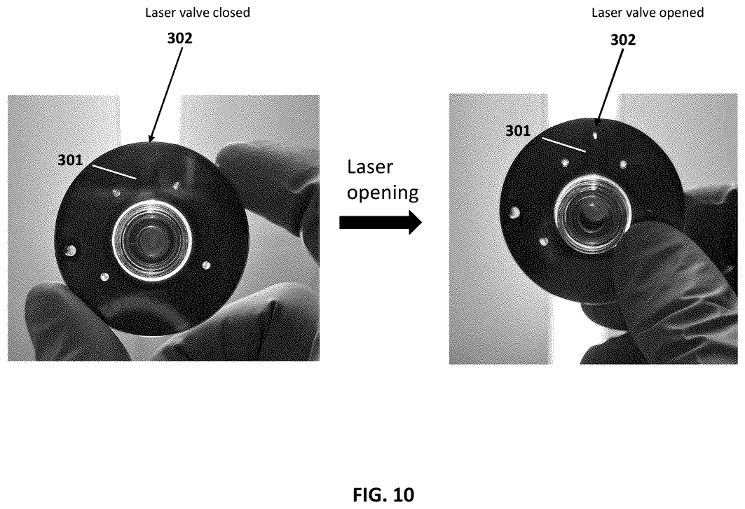

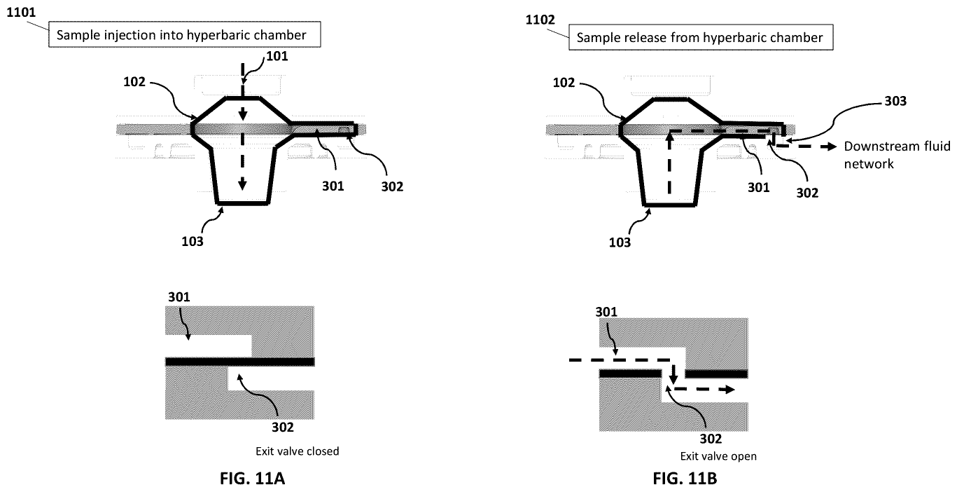

In some embodiments, closed chamber is opened at some time point to release the sample from the heating vessel. The closed chamber may be opened after hyperbaric heating. In some embodiments, the heating chamber is opened using mechanical forces. In some embodiments, the heating chamber is opened by removing a cap, lid, plug, or valve from an outlet. In some embodiments, heating chamber is opened by opening an exit valve 302 at the outlet. For example, the exit valve 302 may be a laser valve that is initially closed and can be opened by shining a laser beam on the valve (FIGS. 10, 11). In some cases, the outlet can be the same as or in the same location as the sample inlet. In other cases, the outlet is in a separate location from the sample inlet.

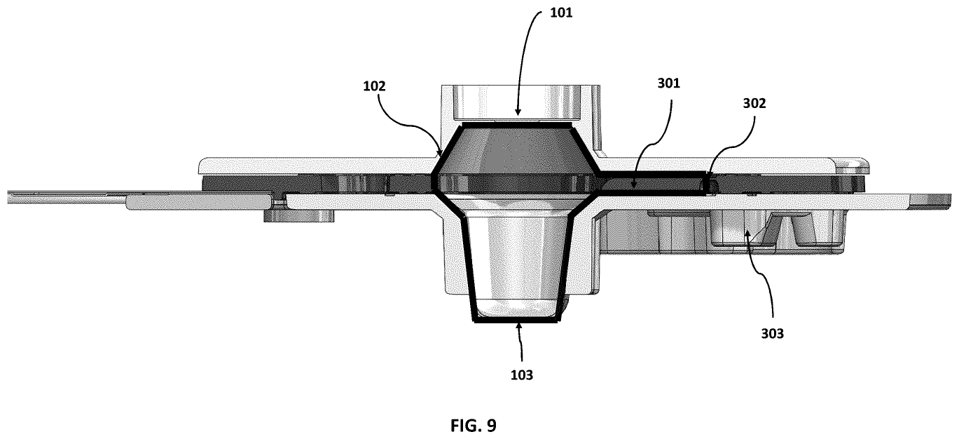

In some embodiments, the sample may be released from an outlet. The outlet may be part of an outlet channel 301 within the heating vessel (FIGS. 9, 10). The outlet channel 301 may comprise or be in contact with the exit valve 302 (FIGS. 9, 10). The outlet channel 301 may be initially sealed by the exit valve 302, for example, during sample injection in operation 1101 in FIG. 11A and during hyperbaric heating. The outlet channel 301 may then be unsealed by opening the exit valve 302, for example, for sample release in operation 1102 in FIG. 11B. When the exit valve 302 is opened, sample may exit the heating chamber through the outlet channel 301. The sample may be propelled out of the heating chamber through the outlet channel 301 using a force, e.g., a centrifugal force. In some embodiments, the sample is released into a collection chamber 303 (FIG. 9). In some embodiments, the collection chamber is a part of the heating vessel. In some embodiments, the collection chamber and heating vessel are part of a fluidics network.

Following release from the hyperbaric heating vessel, the post-hyperbaric heating treatment sample may undergo further sample processing or analysis. The post-hyperbaric heating treatment sample may be used for nucleic acid detection and/or analysis using PCR analysis or a biological (e.g., diagnostic) assay or any of the nucleic acid analysis methods disclosed elsewhere herein.

### CERTAIN EMBODIMENTS OF THE DISCLOSURE

Embodiment 1. A method of analyzing a nucleic acid in a biological sample, the method comprising: heating the biological sample in a closed vessel to a temperature above a boiling point of the biological sample, and analyzing the nucleic acid.

Embodiment 2. A method of analyzing a nucleic acid in a biological sample, the method comprising: heating the biological sample to a temperature of at least 101 degrees Celsius in a closed vessel and analyzing the nucleic acid.

Embodiment 3. A method of analyzing a nucleic acid in a biological sample, the method comprising: heating the biological sample comprising the nucleotide in a closed vessel thereby generating a pressure inside of the vessel between 1 to 200 PSI over atmosphere, and analyzing the nucleic acid.

Embodiment 4. A method of analyzing a nucleic acid in a biological sample, comprising heating the biological sample under hyperbaric conditions, and analyzing the nucleic acid.

Embodiment 5. A method of improving efficiency of molecular amplification comprising: heating a biological sample in a closed vessel thereby generating a pressure inside of the vessel between 10 to 100 PSI over atmosphere, wherein the biological sample comprises a nucleic acid.

Embodiment 6. A method of inactivating molecular amplification inhibitors in a biological sample comprising a nucleic acid, the method comprising: heating the biological sample to a temperature above a boiling point of the biological sample, wherein the nucleic acid is not substantially degraded.

Embodiment 7. The method of embodiment 5 or embodiment 6 wherein the method further comprises analyzing the nucleic acid.

Embodiment 8. The method of any one of embodiments 1 to 7, wherein the analyzing of the nucleic acid comprises one or more of the following: detecting the nucleic acid; sequencing the nucleic acid, and genotyping the nucleic acid.

Embodiment 9. The method of any one of embodiments 1 to 8, wherein the method further comprises detecting the nucleic acid via fluorescence detection.

Embodiment 10. The method of any one of embodiments 1 to 9, wherein the method further comprises amplifying the nucleic acid using molecular amplification.

Embodiment 11. The method of embodiment 10, wherein the method further comprises amplifying the nucleic acid using molecular amplification prior to analyzing the nucleic acid.

Embodiment 12. The method of embodiment 11, wherein the method further comprises amplifying the nucleic acid using molecular amplification after heating the biological sample.

Embodiment 13. A method of analyzing a nucleic acid in a biological sample, comprising: heating the biological sample to in a closed vessel; generating a pressure in the closed vessel above 1 atm; amplifying the nucleic acid using molecular amplification; and analyzing the nucleic acid.

Embodiment 14. A method of analyzing a nucleic acid in a biological sample, comprising: heating the biological sample to a temperature above the boiling point of the biological sample under hyperbaric conditions; amplifying the nucleic acid using molecular amplification; and analyzing the nucleic acid.

Embodiment 15. The method of any one of embodiments 1 to 14, wherein the heating of the biological sample happens for a first time period.

Embodiment 16. The method of embodiment 15, wherein the first time period is between 30 seconds and 3 minutes.

Embodiment 17. The method of any one of embodiments 10 to 16, wherein the molecular amplification comprises polymerase chain reaction (“PCR”).

Embodiment 18. The method of embodiment 17, wherein the analyzing of the nucleic acid comprises detection of a PCR product formed during molecular amplification.

Embodiment 19. The method of any one of embodiments 1 to 18, wherein the biological sample further comprises an additive.

Embodiment 20. The method of embodiment 19, wherein the additive is selected from a chelating agent, a reducing agent, or a nuclease inhibitor.

Embodiment 21. The method of embodiment 20, wherein the additive comprises both a chelating agent and a reducing agent.

Embodiment 22. The method of embodiment 19 or embodiment 20, wherein the additive is a chelating agent.

Embodiment 23. The method of any one of embodiments 20 to 22, wherein the chelating agent is an insoluble chelating agent.

Embodiment 24. The method of embodiment 23, wherein the insoluble chelating agent comprises Chelex.

Embodiment 25. The method of any one of embodiments 20 to 22, wherein the chelating agent is a soluble chelating agent.

Embodiment 26. The method of embodiment 25, wherein the soluble chelating agent comprises EDTA.

Embodiment 27. The method of embodiment 19 or embodiment 20, wherein the additive is a reducing agent.

Embodiment 28. The method of any one of embodiments 19 to 27, wherein the additive is added to the biological sample before the heating step.

Embodiment 29. The method of any one of embodiments 1 to Error! Reference source not found, wherein the biological sample further comprises a single stranded binding protein.

Embodiment 30. The method of any one of embodiments 1 to 29, wherein the nucleic acid is analyzed after the nucleic acid is amplified using between 10 and 45 molecular amplification cycles.

Embodiment 31. The method of any one of embodiments 1 to 30, wherein the nucleic acid is detectable following a lower number of molecular amplification cycles than would be required in the absence of heating.

Embodiment 32. The method of any one of embodiments 1 to 31, wherein the nucleic acid is detectable following a lower number of molecular amplification cycles than would be required as compared to heating the sample at a temperature below the boiling point of the sample for the same time.

Embodiment 33. The method of any one of embodiments 1 to 32, wherein the nucleic acid is detectable following between 20 and 45 molecular amplification cycles.

Embodiment 34. The method of embodiment 33, wherein the nucleic acid is detectable following between 28 and 35 molecular amplification cycles.

Embodiment 35. The method of any one of embodiment 1 to 34, wherein the analyzing of the nucleic acid occurs simultaneously with the molecular amplification of the nucleic acid.

Embodiment 36. The method of any one of embodiment 1 to 34, wherein the analyzing of the nucleic acid and the molecular amplification of the nucleic acid occur sequentially.

Embodiment 37. The method of any one of embodiments 1 to 35, wherein the analyzing of the nucleic acid occurs after each cycle of molecular amplification.

Embodiment 38. The method of any one of embodiments 1 to 37, wherein the biological sample further comprises a plurality of molecular amplification inhibitors and at least 70% of the plurality of molecular amplification inhibitors are inactivated after heating.

Embodiment 39. The method of any one of embodiments 1 to 37, wherein the biological sample further comprises a plurality of molecular amplification inhibitors and at least 90% of the plurality of molecular amplification inhibitors are inactivated after heating.

Embodiment 40. The method of any one of embodiments 1 to 39, wherein the nucleic acid is not substantially degraded after heating.

Embodiment 41. The method of any one of embodiments 1 to 2, 6 to 12, and 14 to 40, wherein the temperature is at least 120 degrees Celsius.

Embodiment 42. The method of any one of embodiments 1 to 2, 6 to 12, and 14 to 40, wherein the temperature is between 101 degrees Celsius and 160 degrees Celsius.

Embodiment 43. The method of any one of embodiments 1 to 2, 6 to 12, and 14 to 40, wherein the temperature is between 120 degrees Celsius and 140 degrees Celsius.

Embodiment 44. The method of any one of embodiments 1 to 2, 6 to 12, and 14 to 40, wherein the temperature is about 130 degrees Celsius.

Embodiment 45. The method of any one of embodiments 1 to 44, wherein the biological sample is heated in an air-tight sealed vessel.

Embodiment 46. The method of embodiment 45, wherein heating of the biological sample generates a pressure of between 10 to 100 PSI inside the air-tight sealed vessel.

Embodiment 47. The method of any one of embodiments 1 to 46, wherein the biological sample comprises a pH of from about 8.0 to about 12.0.

Embodiment 48. The method of any one of embodiments 1 to 47, wherein the nucleic acid is DNA.

Embodiment 49. The method of any one of embodiments 1 to 47, wherein the nucleic acid is RNA.

Embodiment 50. The method of any one of embodiments 1 to 49, wherein the nucleic acid is selected from a viral nucleic acid, a bacterial nucleic acid, a protozoan nucleic acid, and a fungal nucleic acid.

Embodiment 51. The method of embodiment 50, wherein the nucleic acid is a viral nucleic acid.

Embodiment 52. The method of any one of embodiments 1 to 51, wherein the biological sample comprises between 50 copies/mL and 10⁹ copies/mL of the nucleic acid.

Embodiment 53. The method of any one of embodiments 1 to 52, wherein a volume of the biological sample is between 100 μL and 5 mL.

Embodiment 54. The method of any one of embodiments 1 to 53, wherein the biological sample comprises a pathogen or portion thereof.

Embodiment 55. The method of embodiment 54, wherein the pathogen or portion thereof is selected from a virus or a portion thereof, a bacterium or a portion thereof, a protozoon or a portion thereof, a yeast or a portion thereof, and a fungus or a portion thereof.

Embodiment 56. The method of embodiment 55, wherein the pathogen is a virus or a portion thereof.

Embodiment 57. The method of embodiment 56, wherein the virus is SARS-CoV2.

Embodiment 58. The method of any one of embodiments 1 to 57, wherein the biological sample comprises a substance selected from blood, lacrimal fluid, saliva, mucus, sputum, feces, cerebrospinal fluid and urine.

Embodiment 59. The method of embodiment 58, wherein the biological sample comprises mucus.

Embodiment 60. The method of embodiment 59, wherein the biological sample is collected via nasopharyngeal swab, cervical swab, or nasal swab from a subject.

Embodiment 61. The method of any one of embodiments 1 to 60, wherein the biological sample is heated with a heat source.

Embodiment 62. The method of embodiment 61, wherein the heat source comprises an induction heater, a heating element, or a microwave.

Embodiment 63. The method of any one of embodiments 1 to 2, 6 to 12, or 14 to 62, wherein the biological sample remains at the temperature for between 1 second and 300 seconds.

Embodiment 64. The method of any one of embodiments 1 to 2, 6 to 12, or 14 to 62, wherein the biological sample remains at the temperature for between 30 second and 120 seconds.

Embodiment 65. The method of any one of embodiments 1 to 64, wherein the biological sample was collected from a subject.

Embodiment 66. The method of embodiment 65, wherein the subject is a human.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are included for illustrative purposes only and are not intended to limit the scope of the invention.

### Example 1: Hyperbaric Heating Method Efficiently Neutralizes Rt-PCR Inhibitor

In this example, methods of the present disclosure were used to prepare samples for RT-PCR, and these methods were compared with a standard method. The efficiency of RT-PCR (denoted by average cycle threshold) was compared from the two methods of sample preparation.

### Material Used

Cryogenic tube; metallic paramagnetic zinc coated 6 mm disc; 1.5 ml collection buffer composed of 10 mm Tris/EDTA, 1 mM NaCl; 10% W/v Chelex 100 resin; CDC SARS-COV2 RT-PCR primers; Puritan swab.

### Experimental Procedure

Nasal specimens were collected using Puritan swab and nasal matrix was resuspended in 1.5 ml collection buffer. Next, 250 microliters of the nasal specimen resuspended in collection buffer was placed into a cryogenic tube having two metallic discs at their bottom and having 10% Chelex 100. To the cryogenic tubes were added 32000 copies per milliliter of inactivated SARS-COV2 viruses.

For methods of the present disclosure, cryogenic tubes were then placed under an oscillating magnetic field generated via an induction coil and then were hyperbarically heated for 40, 60, 80 and 100 seconds, reaching 100° C., 103° C., 108° C., and 110° C., respectively. For control, the exact same sample in the same buffer and using the same Chelex percentage was heated in a heat block at 98° C. for 5 minutes.

### Results

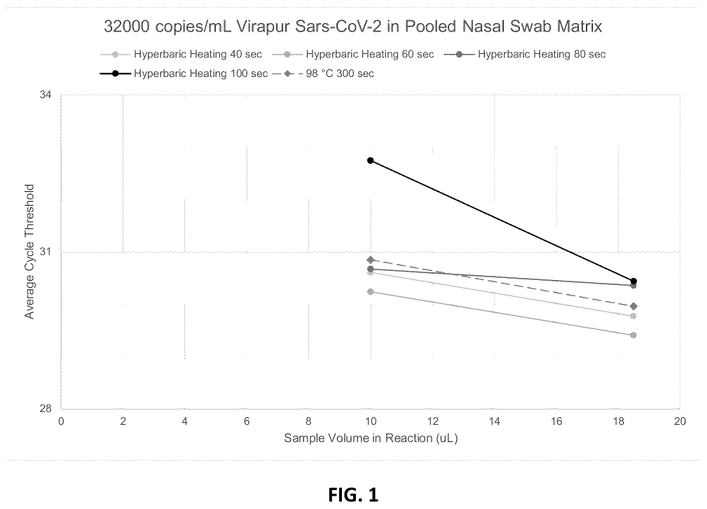

FIG. 1 shows the results of the RT-PCR experiment following condition described in the experimental procedure (hyperbaric heating at or above 100 degrees Celsius for 40, 60, 80 and 100 seconds) and the standard condition of heating at 98 degrees Celsius for 300 seconds. Each sample preparation method was performed on a 10 μL sample and a 20 μL sample to assess any effect from sample volume. All conditions tested efficiently neutralized PCR inhibitors from the nasal specimen, indicated by an average cycle threshold of 33 or less. Moreover, hyperbaric heating the 10 μL sample above 100 degrees Celsius for 40, 60, and 80 seconds resulted in an average cycle threshold of 31 or less, comparable to heating for 300 seconds and 98 degrees Celsius. Further, for the 20 μL sample, an average cycle threshold of less than 31 was achieved for all tested conditions, including hyperbaric heating above 100 degrees Celsius. Thus, adding more volume to the preparation increases the sensitivity of the PCR, and there is no reactivation of PCR inhibitors in the sample preparation.

The results set forth in FIG. 1 demonstrate that the method of the present disclosure gives superior results than traditional method of heating at 95-98 degrees Celsius. For example, 60 second of hyperbaric heating reaching 103 degrees Celsius is clearly superior to the standard conditions of 98 degrees Celsius for 300 seconds, providing a lower average cycle threshold in one-fifth of the time.

### Example 2: Effect of Temperature for Sample Preparation on RT-PCR Efficiency

In this example, the effect of temperature on sample preparation was tested. RT-PCR was run to determine the efficiency of the sample preparation at different temperatures (95° C., 105° C., 120° C., and 130° C.).

### Material Used

Glass ampule; 10% Chelex 100; collection buffer: 10 mm Tris/EDTA, 1 mM NaCl, 0.02% azide; 1 micromolar of thermostable single stranded nucleic acid binding protein; heating block with aluminum beads; CDC Sars-CoV2 RT-PCR primers; QiaAmp viral RNA kit.

### Experimental Procedure

The heating block was set-up at 95° C., 105° C., 120° C., and 130° C.). 250 microliter of nasal matrix was resuspended in the collection buffer and added 32000 copies of inactivated SARS-COV2. This 250 microliter of sample was then placed in a glass ampule before being gas-tight sealed. Tubes were placed in heat block set at the appropriate temperature for 2 minutes. Supernatant were collected and RT-PCR were run on commercial thermocycler. For control, exact sample in the same buffer was also tested at the same temperatures.

### Results

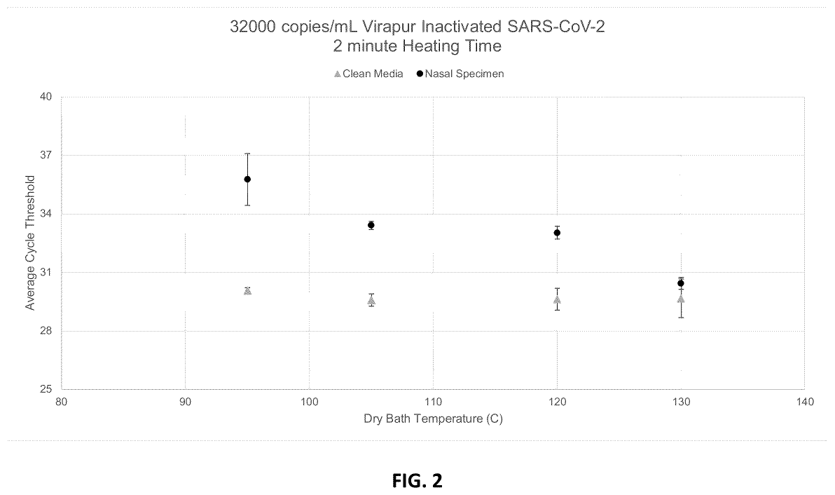

FIG. 2 depicts the average cycle threshold for the detection of inactivated SARS-CoV-2 in (1) a simple buffer solution (dark dots on graph) and (2) a swab sample. Each of these samples was subjected to different temperatures (95° C., 105° C., 120° C., and 130° C.) for 2 minutes.

The results set forth in FIG. 2 demonstrate that the method of the present disclosure provides efficient neutralization of PCR inhibitors. As seen in FIG. 2, the average cycle threshold for the swab and buffer samples heated at 95° C. are 36 and 30, respectively. The higher average cycle threshold for the swab sample (light grey dot, top left) compared to the buffer sample (dark dot, bottom left), is indicative of PCR inhibitors remaining in the nasal sample following heating that are not present in the buffer solution. Thus, the standard method of heating at 95° C. was not able to inactivate all PCR inhibitors in the nasal swab sample after 2 minutes. In contrast, after hyperbaric heating at 130° C. for 2 minutes, both the nasal swab sample (light grey dot, right) and the buffer sample (dark dot, right) exhibited essentially the same average cycle threshold (average cycle threshold of about 30), indicating nearly complete inactivation of all PCR inhibitors present in the nasal swab sample.

Further, the results depicted in FIG. 2 demonstrate that the method of the present disclosure does not cause detectable nucleic acid degradation. The average cycle threshold for the buffer sample was consistent (average cycle threshold of about 30) across all temperatures examined, demonstrating that hyperbaric heating the buffer sample to temperatures as high as 130° C. did not cause significant nucleic acid degradation in the buffer sample. Moreover, no appreciable degradation was identified in the nasal swab sample (light grey dot, right), as it exhibited the same average cycle threshold as the buffer sample (dark dot, right). In view of FIG. 2, it is evident that the method of the present disclosure achieves efficient inactivation of PCR inhibitors without degrading nucleic acid.

### Example 3: Hyperbaric Heating Method Matches Industry Standard Procedure Regarding Time Required for PCR Amplification

In this example, a method of the present disclosure was compared to a standard sample preparation method using QiaAmp viral RNA kit, and PCR amplification efficiency for each method was assessed at different concentrations of analyte (SARS-CoV2 copies).

### Material Used

Glass ampule; 10% Chelex 100; collection buffer: 10 mm Tris/EDTA, 1 mM NaCl, 0.02% azide; 1 micromolar of thermostable single stranded nucleic acid binding protein; heating block with aluminum beads; CDC Sars-CoV2 RT-PCR primers; QiaAmp viral RNA kit.

### Experimental Procedure

Several aliquots of 250 microliter of nasal matrix were resuspended in collection buffer and to each aliquot was added a different concentration of inactivated SARS-CoV2 (200, 500, 1000, 2000, 4000, 8000, 16000 and 32000 copies per milliliter of inactivated SARS-CoV2).

For the method of the present disclosure (FAST PCR Prep), the heating block was set at 130 degrees Celsius. Each 250 microliter of sample was then placed in a glass ampule before being gas-tight sealed. Tubes were placed at 130 degrees Celsius for 2 minutes. Supernatant were collected and RT-PCR were run on commercial thermocycler.

In parallel, as a control, a second copy of the sample aliquots having the same copies per milliliter concentration of SARS-CoV2 were purified using QiaAmp viral RNA kit pursuant to the manufacturer's instructions. Each experimental group was performed in duplicate.

### Results

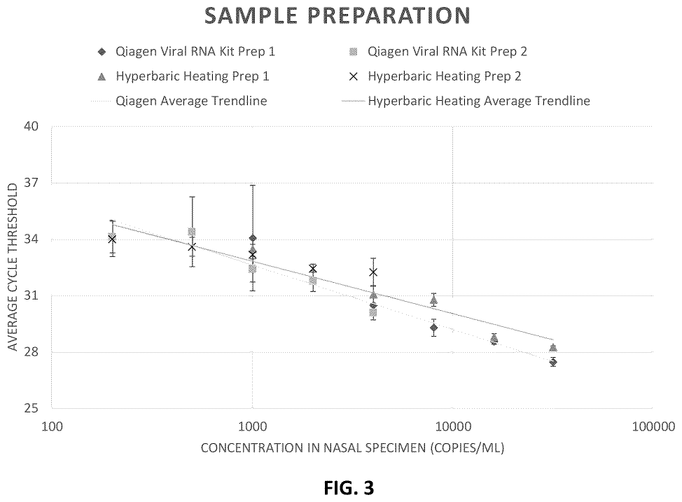

FIG. 3 shows that the average cycle threshold from a method of the present disclosure (labeled as “FASTPCR Superheating Prep) is similar to the average cycle threshold obtained from sample preparation using Qiagen Viral RNA Kit prep. The similar average cycle threshold value between the two methods was observed across multiple concentrations of analyte in nasal specimens, with higher analyte concentrations resulting in lower average cycle threshold values for each method. The results set forth in FIG. 3 thereby demonstrate that PCR efficiency of a method of the present disclosure matches the performance of the Qiagen Viral RNA Kit for the detection of SARS-COV2 in significantly less time.

### Example 4: Hyperbaric Heating Method in Absence of Chelating Agent

In this example, different agents such as RNAse inhibitor and/or reducing agent were tested in sample preparation for RT-PCR using a method of the present disclosure.

### Material Used

Stainless steel cup; collection buffer comprising 10 mm Tris/EDTA, 1 mM NaCl; induction heating system; and respiratory syncytial virus (RSV) RT-PCR primers.

### Experimental Procedure

A 250 μL sample of nasal swab matrix was collected and resuspended in collection buffer. To the mixture of collection and nasal swab matrix was added with 32000 copies of inactivated respiratory syncytial virus (RSV). Prior to heating, to some aliquots of the sample was added (a) nothing, (b) 1 mM DTT, (c) 200 U of RNAse (Ambion) alone, or (d) combination of both 1 mM DTT and 200 U of RNAse. The 250 μL of sample was then placed in a stainless-steel vessel before being gas-tight sealed. Next, the vessel was placed in induction heating system set at the 150° C. measured at the cup surface for 20 seconds. After the hyperbaric heating step, supernatants from each sample were collected and RT-PCR were run on commercial thermocycler.

For control, exact same sample were tested in clean buffer using same conditions.

### Results

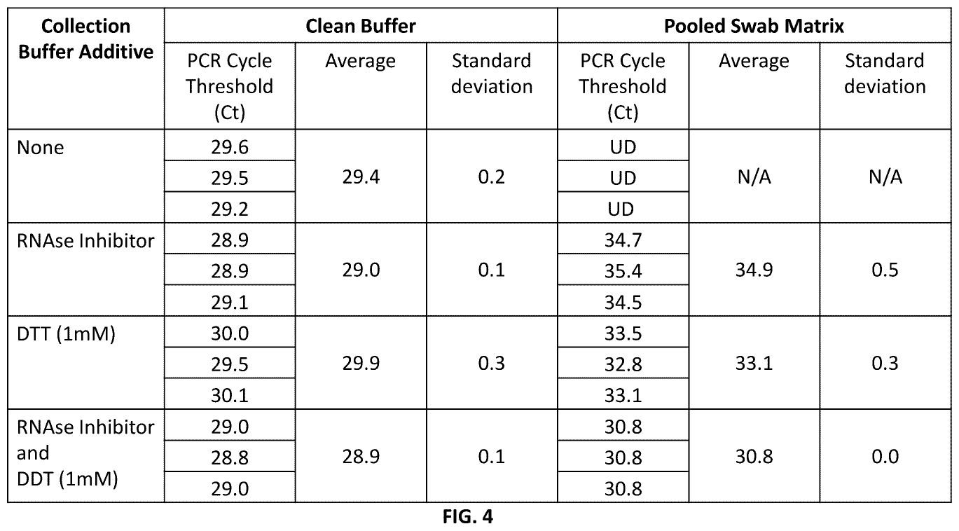

FIG. 4 shows a table of the average cycle threshold for the detection of inactivated RSV RNA in both clean buffer and pooled swab under four conditions: (1) no additive (first row); (2) 200 U of RNAse inhibitor only (second row); (3) 1 mM DTT only (third row); and (4) both 1 mM DTT and 200 U RNAse inhibitor (forth row). A chelating agent was not added in any of these testing groups. UD indicated undetected and N/A indicates not-applicable.

The result shows that, in the absence of Chelex, sample preparation of pooled swab matrix using hyperbaric heating is at least effective in the presence of an RNAse inhibitor or a reducing agent (such as 1 mM DDT) (average cycle thresholds of 34.9 and 33.1, respectively). Combination of reducing agent and RNAse inhibitor in the collection buffer provides even better results for sample preparation of pooled swab matrix using a hyperbaric heating method. The average cycle of RT-PCR from the sample preparation using both reducing agent and an RNAse inhibitor was similar to control sample using clear buffer (average cycle thresholds of 30.8 and 28.9, respectively), indicating nearly complete inactivation of PCR inhibitors in the pooled swab matrix sample.

According to results in FIG. 4, hyperbaric heating method for sample preparation provides efficient neutralization of PCR inhibitors even in the absence of Chelex. This method is particularly effectively in the presence of a reducing agent such as DDT, an RNAse inhibitor, and/or combination of a reducing agent and an RNAse inhibitor. Additionally, the results set forth in FIG. 4 shows nearly complete neutralization of PCR inhibitors when RNAse was added in combination with the reducing agent.

Thus, even in the absence of chelating agent, a hyperbaric heating method of the present disclosure is effective for sample preparation (e.g., inactivation of PCR inhibitors), particularly in the presence of an additive (e.g., a reducing agent, an RNAse inhibitors, and/or a combination thereof).

### Example 5: Hyperbaric Heating Across Multiple Sample Sources

In this example, a method of the present disclosure was shown to provide similar PCR detection and sensitivity for a variety of nucleic acid containing samples as compared to a standard sample preparation method using Qiagen Viral RNA kit or Qiagen DNeasy UltraClean Microbe DNA Kit.

### Material Used

Metallic heating vessel; induction coil; collection buffer (1×TE Buffer and 2 mg/mL BSA); Chelex-100 Chelating Resin (final concentration in treatment sample prior to hyperbaric heating: 12% weight/volume of treatment sample); DTT (final concentration in treatment sample prior to hyperbaric heating: 1 mM); thermostable Thermococcus kodakarensis (KOD) (final concentration in treatment sample prior to hyperbaric heating: 0.5 μM); nucleic acid containing samples; centrifuge; PCR primers targeting the nucleic acid in the nucleic acid containing samples; Qiagen Viral RNA kit; Qiagen DNeasy UltraClean Microbe DNA Kit. The treatment sample is the mixture of the nucleic acid-containing sample, collection buffer, Chelex-100 Chelating Resin, Thermococcus kodakarensis (KOD), and DTT.

### Nucleic Acid Containing Samples

The following nucleic acid containing samples were tested in this example: SARS-CoV-2, HeLa Mammalian Cells, S. cerevisiae Yeast, B. cereus Bacterial Spore, B. subtilis Bacteria, and C. albicans Fungi.

### Experimental Procedure

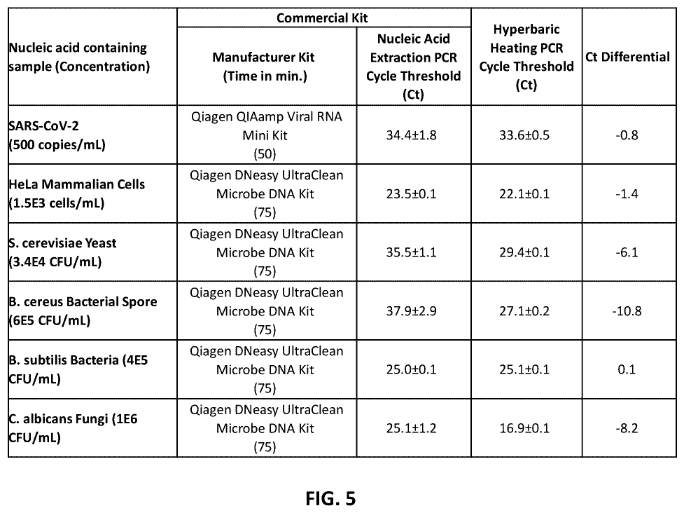

The nucleic containing samples were first prepared PCR by a hyperbaric heating method. First, the nucleic containing samples were individually diluted using collection buffer (1×TE Buffer and 2 mg/mL BSA). The concentrations of the diluted nucleic acid sample in collection buffer are shown in FIG. 5. For each sample, the metallic heating vessel was prepared with the following reagent components: Chelex-100 Chelating Resin (48 mg), DTT (3.2 μL), and Thermococcus kodakarensis (KOD) (8 μL), BSA (2 mg/mL). For each run, one of the diluted nucleic acid containing samples (388.8 uL) was pipetted into the metallic heating vessel containing the Chelex-100 Chelating Resin, Thermococcus kodakarensis, and DTT. The metallic heating vessel was sealed with an airtight cap and placed within the induction coil and heated for 15 seconds. The metallic heating vessel was cooled at ambient temperature until safe to handle. The metallic heating vessel was placed in a centrifuge and spun to collect the material at the bottom of the vessel. The metallic heating was then opened and the sample was aspirated into a microcentrifuge tube. The microcentrifuge tube was spun to pellet the Chelex-100 Chelating Resin, and the supernatant was collected for PCR analysis.

In parallel, control samples were also prepared from the original diluted nucleic acid sample aliquots. These samples not subjected to hyperbaric heating and were purified using a commercial nucleic acid purification kit. For samples containing SARS-CoV-2, a QiaAmp viral RNA kit was used pursuant to the manufacturer's instructions. For the samples containing HeLa Mammalian cells, S. cerevisiae yeast, B. cereus bacterial spore, B. subtilis bacteria, and C. albicans fungi, a Qiagen DNeasy UltraClean Microbe DNA Kit was used to purify the nucleic acid, pursuant to the manufacturer's instructions. The microbe DNA extraction protocol required 1.8 mL of sample and was performed in 70 minutes.

The samples prepared by hyperbaric heating and the control samples prepared by the commercial purification kit were then used for PCR analysis. PCR amplification was performed on the Applied Biosystems Quantstudio 3 and Quantstudio 5 PCR instruments. Each experimental group was performed in duplicate.

### Results

The results showed that that the average cycle threshold from a method of the present disclosure (labeled as “hyperbaric heating”) is similar to the average cycle threshold obtained from sample preparation using the Qiagen Viral RNA Kit prep or the Qiagen DNeasy UltraClean Microbe DNA Kit across different nucleic acid containing samples. FIG. 5 shows a table of the average cycle threshold for the detection of inactivated nucleic acid sample from each sample source. The 15-second hyperbaric heating yielded equivalent (within error range) or faster Ct values for each sample. The results set forth in FIG. 5 thereby demonstrate that PCR efficiency of a method of the present disclosure matches the performance of the commercial for the detection of nucleic acid from multiple sample sources, including SARS-CoV-2, mammalian cells, yeast, bacteria, and fungi, in significantly less time.

### Example 6: Modifying Temperature Ramp Rate on Nucleic Acid Sample Preparation by Hyperbaric Heating

In this example, different hyperbaric heating vessels were created for hyperbaric heating with different heating temperature ramp rates. The temperature was estimated using an internal temperature sensor inside the closed vessel and correlated via an external temperature infrared sensor assessing the exterior temperature of the closed vessel. Table 1 shows different heating vessels that were created and methods of heating (hyperbaric heating time) and the heating temperature ramp rate for each vessel.

TABLE 1
Hyperbaric heating temperature ramp rates

| | Heating temperature | Hyperbaric heating |
|---|---|---|
| Hyperbaric heating vessel | ramp rate (° C. / s) | time (s) |
| Glass ampule - non | 1 | 120 |
| induction heating | | |
| Pressure cooker 1 with | 2.27 | 80 |
| Zinc disc - induction | | |
| heating | | |
| Pressure cooker 1 with | 2.86 | 65 |
| stainless steel disc - | | |
| induction heating | | |
| Bullet | 6.14 | 20 |
| Pressure cooker 1.5 | 8 | 17 |
| Pressure cooker 2 | 12 | 15 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method of analyzing a nucleic acid, the method comprising:
(a) heating a treatment sample comprising a nucleic acid and a molecular amplification inhibitor in a closed heating chamber in a hyperbaric condition from a first temperature to a second temperature over a ramp time, wherein said second temperature is selected from 106 degrees Celsius to 160 degrees Celsius, thereby producing a heat-treated sample comprising said nucleic acid; and
(b) analyzing said nucleic acid from said heat-treated sample.

2. The method of claim 1, wherein said treatment sample comprises one or more reagents selected from: a chelating agent, a single stranded nucleic acid binding protein, a reducing agent, and a stabilizer.

3. The method of claim 2, wherein said treatment sample comprises a chelating agent, a single stranded nucleic acid binding protein, and a reducing agent.

4. The method of claim 3, wherein said treatment sample comprises a chelating agent, a single stranded nucleic acid binding protein, a reducing agent, and a stabilizer.

5. The method of claim 4, wherein said stabilizer is selected from bovine serum albumin and gelatin.

6. The method of claim 3, wherein said treatment sample further comprises a protease or a nuclease inhibitor.

7. The method of claim 1, wherein said ramp time is selected from 3 to 50 seconds.

8. The method of claim 1, wherein said heating in (a) occurs at a temperature ramp rate from 5 degrees Celsius per second to 50 degrees Celsius per second.

9. The method of claim 1, further comprising, after said heating in (a) and prior to said analyzing in (b), maintaining said heat-treated sample at said second temperature for a maintenance time from 5 seconds to 120 seconds prior to cooling said heat-treated sample.

10. The method of claim 1, further comprising, after said heating in (a) and prior to said analyzing in (b), cooling said heat-treated sample to ambient temperature.

11. The method of claim 1, wherein said heating in (a) comprises induction heating.

12. The method of claim 1, wherein said analyzing in (b) comprises amplifying said nucleic acid by polymerase chain reaction (PCR), thereby producing a PCR product.

13. The method of claim 12, wherein said PCR product is detectable after amplifying said nucleic acid from 10 to 55 molecular amplification cycles.

14. The method of claim 12, wherein said PCR product is detectable after amplifying said nucleic acid from 28 to 35 molecular amplification cycles.

15. The method of claim 12, wherein said analyzing in (b) further comprises sequencing said PCR product after said amplifying.

16. The method of claim 1, wherein said analyzing in (b) comprises sequencing said nucleic acid.

17. The method of claim 1, wherein said nucleic acid is not extracted, isolated, or otherwise purified from said heat-treated sample prior to said analyzing in (b).

18. The method of claim 1, wherein said nucleic acid is DNA.

19. The method of claim 1, wherein said nucleic acid is RNA.

20. The method of claim 1, wherein said heating inactivates at least 60% of said molecular amplification inhibitor.

21. The method of claim 1, wherein said molecular amplification inhibitor comprises (i) an agent that binds to said nucleic acid, (ii) a DNAse, or (iii) an RNase.

22. The method of claim 1, wherein said treatment sample comprises a bodily sample comprising said nucleic acid, wherein:
i) said bodily sample is selected from the group consisting of a blood sample, a lacrimal fluid sample, a saliva sample, a mucus sample, a sputum sample, a feces sample, a cerebrospinal fluid sample, and a urine sample; and
ii) said nucleic acid is not extracted, isolated, or otherwise purified from said bodily sample.

23. A method of analyzing a nucleic acid, the method comprising:
(a) heating a treatment sample comprising a nucleic acid and a molecular amplification inhibitor in a closed heating chamber in a hyperbaric condition from a first temperature to a second temperature above 100 degrees Celsius, thereby producing a heat-treated sample; and
(b) analyzing said nucleic acid from said heat-treated sample;
wherein said treatment sample comprises one or more reagents selected from the group consisting of a chelating agent, a single stranded nucleic acid binding protein, a reducing agent, and a stabilizer.
